(19) United States
(12) Patent Application Publication
(10) Pub. No.: US 2018/0161291 A1
(43) Pub. Date: Jun. 14, 2018

(54) EXTENDED RELEASE UREA COMPOSITIONS

(71) Applicant: Kato Pharmaceuticals, Inc., Laguna Hills, CA (US)

(72) Inventor: Terrance McGovern, Laguna Hills, CA (US)

(21) Appl. No.: 15/579,552

(22) PCT Filed: Jun. 3, 2016
(86) PCT No.: PCT/US16/35842

§ 371 Date: Dec. 4, 2017

Related U.S. Application Data
(60) Provisional application No. 62/171,888, filed on Jun. 5, 2015.

(51) Int. Cl. A61K 31/17; A61K 47/10; A61K 47/12; A61K 47/02; A61K 47/36; A61K 9/00; A61P 27/02

(52) U.S. Cl. CPC A61K 31/17; A61K 47/10; A61K 47/12; A61P 27/02; A61K 47/36; A61K 9/0024; A61K 9/0048; A61K 47/02

(57) ABSTRACT

Embodiments disclosed herein provide compositions, depot gel injections, and sustained-release drug delivery systems comprising urea and/or urea derivative and uses thereof.

### CROSS-REFERENCE TO RELATED APPLICATIONS

This application claims the benefit of U.S. Provisional Application No. 62/171,888, filed Jun. 5, 2015, the entire content of which is incorporated herein by reference in its entirety for all purposes.

### BACKGROUND

The vitreous humor of a normal human eye is a gel that is roughly 99% water and 1% macromolecules. These macromolecules include a network of collagen fibrils, hyaluronic acid, soluble glycoproteins, sugars and other low molecular weight metabolites. Type II collagen is the principal fibrillar collagen of the vitreous, but the vitreous also contains collagen types V, IX, and XI. The posterior portion of the vitreous body, the posterior hyaloid surface (also known as the posterior vitreous cortex), is in direct contact with the inner retinal surface most prominently at the vitreous base, optic disc, and along the major retinal vessels. Normal adhesion of the vitreous to the retina is mediated by cellular and molecular interactions between the posterior vitreous cortex and the inner limiting membrane (ILM) of the retina. The ILM is essentially the basement membrane of retinal Mueller cells. The ILM contains collagen types I and IV, glycoproteins such as laminin and fibronectin and other glycoconjugates. These components are thought to bridge and bind collagen fibers between the vitreous and the ILM.

With age, the vitreous humor changes from gel to liquid and as it does so it gradually shrinks and separates from the ILM of the retina. This process is known as “posterior vitreous detachment” (PVD) and is a normal occurrence after age 40. However, degenerative changes in the vitreous may also be induced by pathological conditions such as diabetes, Eales disease and uveitis. Also, PVD may occur earlier than normal in nearsighted people and in those who have had cataract surgery. Usually, the vitreous makes a clean break from the retina. Occasionally, however, the vitreous adheres tightly to the retina in certain places. These small foci of resisting, abnormally firm attachments of the vitreous can transmit great tractional forces from the vitreous to the retina at the attachment site. This persistent tugging by the vitreous often results in horseshoe-shaped tears in the retina. Unless the retinal tears are repaired, vitreous fluid can seep through this tear into or underneath the retina and cause a retinal detachment, a very serious, sight-threatening condition. In addition, persistent attachment between the vitreous and the ILM can result in bleeding from rupture of blood vessels, which results in the clouding and opacification of the vitreous.

The development of an incomplete PVD has an impact on many vitreoretinal diseases including vitreomacular traction syndrome, vitreous hemorrhage, macular holes, macular edema, diabetic retinopathy, diabetic maculopathy and retinal detachment. Thus, an important goal of vitreous surgery is to separate the vitreous from the retina in a manner that prevents vitreous traction.

In order to remove the vitreous from the eye, a microsurgical procedure called vitrectomy is usually performed. In this procedure the vitreous is removed from the eye with a miniature handheld cutting device while simultaneously replacing the removed vitreous with saline solution to prevent collapse of the eye. Surgical removal of the vitreous using this method is highly skill-dependent, and complete removal of the cortical vitreous remains a difficult task. Furthermore, mechanical vitrectomy carries the risk of complications such as scarring, tearing and other damage to the retina. Obviously, such damage is highly undesirable as it can compromise the patient's vision after surgery.

Thus, alternative methods to remove the vitreous from the retina have been the focus of recent investigation. Such methods have explored the use of enzymes and chemical substances, which can be used to induce/promote liquefaction of the vitreous and/or separation of the vitreoretinal interface (PVD). These approaches, which are referred to as “pharmacological vitrectomy,” have included several proteolytic enzymes such as alpha-chymotrypsin, hyaluronidase, bacterial collagenase, chondroitinase and dispase, which have been injected intravitreally in experimental and/or clinical trials to induce PVD. However, most of these techniques do not release the posterior hyaloid from the ILM completely or without complications. In addition, in several of these cases, the risk of adverse reactions is high. For example, the use of bacterial proteases in mammalian systems generates an immune response, which leads to proliferative vitreoretinopathy resulting in complex retinal re-detachment. Collagenase has been reported to liquefy the vitreous, but it has also been shown to disrupt the outer layers of the retina. Alpha-chymotrypsin has been reported to produce peripapillary and vitreous hemorrhage in the injected eyes. Finally, dispase has been reported to cause toxicity to the inner layer of the retina 15 minutes after injection. Depending on the concentration of dispase used, proliferative retinopathy or epiretinal cellular membranes can develop in the injected eyes.

Urea and urea derivatives have also been used to liquefy the vitreous humor and induce a posterior vitreous detachment via intravitreal injection. See e.g., U.S. Pat. Nos. 6,462,071, 7,008,960, 7,977,385 and 8,691,874; the entire disclosures of which are incorporated herein by reference thereto.

U.S. Pat. No. 5,470,881 (Charlton et al.), U.S. Pat. No. 5,629,344 (Charlton et al.) have described the topical application to the cornea or the “surface” of the eye of urea and/or urea derivatives to treat ocular conditions such as dryness, non-infectious keratitis, irregularities of the corneal or conjunctival epithelium, ocular scarring and “subjective irritations” of the eye. It is important to note that the urea formulations that have been described in the above mentioned patents utilize formulations which are non-aqueous in nature. These formulations contain hydrophobic non-aqueous systems like white petrolatum, mineral oil, and anhydrous liquid lanolin. Some of the formulations described are aqueous in nature; however, in place of urea the authors suggest the use of urea derivatives like ureidopropionic acid, or allantoin.

In the past, some aqueous urea preparations were reported to hydrolyze, thus producing ammonia as a byproduct. Ammonia is toxic to the eye when applied topically, and is even more toxic when applied intravitreally.

Urea is a small molecule having a molecular weight of 60.06. Urea is somewhat basic, the pH of a 10% water solution is 7.2. Urea is very soluble in water, ethanol, methanol and glycerol; however, it is practically insoluble in chloroform or ether. Urea is colorless to white, prismatic crystals or white crystalline powder which stored under dry conditions is stable at room temperature. Aqueous urea solutions freshly prepared are clear, colorless and odorless. However, aqueous urea solutions gradually degrade and develop an odor of ammonia.

Urea is a product of the metabolism of proteins in the human body, it is excreted in human urine in average amounts of 30 gm/day. Urea has been widely used in medicine. Urea Solution for Injection has been an Official Monograph in the United States Pharmacopoeia/National Formulary (USP 24, 2000, pp. 1730), for over 40 years. Urea for injection (intravenous) had been a US Food and Drug approved product for over 20 years. The Urea product was registered and sold in the United States by Abbot Pharmaceutical Co. in 1961, under the trade name of Ureaphil. The Physicians' Desk Reference (PDR, Edition 1961, Medical Economics Publishing) lists the Urea for injection 1961 through 1979. References indicate that Urea for Injection was also registered and sold in numerous International countries as an osmotic diuretic for the reduction of intracranial pressure as well as for the reduction of intraocular pressure in subjects with Glaucoma. (Tartar, R. C. et al. American Journal of Opthalmology: 52:323-331; 1961). In addition, urea has been used intravenously to treat painful crisis of sickle-cell disease. (McCurdy, P. R. I.V. “Urea treatment of the painful crisis of Sickle Cell Disease” New England Journal of Medicine. 285: 992-994; 1971).

Urea has been used topically as a dermatological active ingredient in the treatment of Psoriasis, ichthyosis, atopic dermatitis and removal of excess keratin from dry skin. (Remington. “The Science and Practice of Pharmacy” 19 Edition, Chapter 62, pp. 1041-1042, 1995).

### SUMMARY

Embodiments disclosed herein provide biocompatible, biodegradable compositions for intravitreal injection in the treatment of an eye disorder, the compositions comprising: a hydrogel; and 1.0-99.0% urea by weight.

Embodiments disclosed herein provide pharmaceutical compositions for injection into a vitreous body of an eye, comprising a sterile aqueous solution, comprising: a) 1.0-99.0% urea by weight; b) 0.00001-1.0% citric acid by weight; c) 0.05-3.6% sodium chloride by weight; and d) sterile water for injection, Q.S. 100% by weight; wherein said pharmaceutical composition is stable and non-toxic when injected into the eye; and wherein said pharmaceutical composition is retained within a hydrogel thereby providing extended release.

Embodiments disclosed herein provide methods of treating an eye disorder, comprising: injecting into the vitreous body a composition comprising an extended release hydrogel, comprising PEG and 1.0-99.0% urea by weight. In some embodiments, the eye disorder is selected from VMA, PVD, diabetic retinopathy, diabetic macular edema, and wet macular degeneration.

Embodiments disclosed herein provide methods of treating an eye disorder, comprising: encapsulating an extended release hydrogel comprising PEG and 1.0-99.0% urea by weight to form a microencapsulation; and injecting into the vitreous body with the microencapsulation.

Embodiments disclosed herein provide depot gel injections for treating an eye disorder, comprising: a depot gel vehicle comprising a biocompatible polymer, the depot gel vehicle configured to encapsulate a composition comprising 1.0-99.0% urea by weight.

Embodiments disclosed herein provide sustained-release drug delivery systems comprising 1.0-99.0% urea and/or urea derivative by weight encapsulated within a biomaterial. In some embodiments, the sustained-release drug delivery systems comprise an implant. In some embodiments, the implant comprises one or more compartments. In some embodiments, the urea and/or urea derivative and the biomaterial are contained in the compartment. In some embodiments, the biomaterial comprises a polymer. In some embodiments, the urea and the polymer form a composite material. In some embodiments, the composite material comprises a microparticle or nanoparticle. In some embodiments, the microparticle or nanoparticle forms multiple layers. In some embodiments, the layers are separated by urea solution and/or crystalline. In some embodiments, the microparticle or nanoparticle comprises a hydrogel. In some embodiments, the hydrogel is a PEG-chitosan hydrogel. In some embodiments, the sustained-release drug delivery systems comprise 10-90% urea and/or urea derivative by weight. In some embodiments, the sustained-release drug delivery systems comprise 10-60% urea and/or urea derivative by weight. In some embodiments, the sustained-release drug delivery systems comprise 20-40% urea and/or urea derivative by weight.

Embodiments disclosed herein provide methods of treating an eye disorder, comprising administering the sustained-release drug delivery systems disclosed herein to a subject. In some embodiments, administering the sustained-release drug delivery system comprises intravitreal injecting the sustained-release drug delivery system to the subject. In some embodiments, administering the sustained-release drug delivery system comprises intravitreal implanting the sustained-release drug delivery system to the subject. In some embodiments, administering the sustained-release drug delivery system comprises administering the sustained-release drug delivery system to the subject by iontophoresis. In some embodiments, administering the sustained-release drug delivery system comprises administering the sustained-release drug delivery system to the subject by a microelectromechanical device. In some embodiments, the eye disorder is selected from the group consisting of VMA, PVD, diabetic retinopathy, diabetic macular edema, and wet macular degeneration. In some embodiments, the sustained-release drug delivery system releases the urea and/or urea derivative for at least 6 months for a single administration. In some embodiments, the sustained-release drug delivery system releases the urea and/or urea derivative for at least 12 months for a single administration. In some embodiments, the sustained-release drug delivery system releases the urea and/or urea derivative for at least 24 months for a single administration. In some embodiments, the sustained-release drug delivery system releases the urea and/or urea derivative for at least 5 days for a single administration. In some embodiments, the sustained-release drug delivery system releases the urea and/or urea derivative for at least 14 days for a single administration. In some embodiments, the sustained-release drug delivery system releases the urea and/or urea derivative for at least 30 days for a single administration.

### BRIEF DESCRIPTION OF THE DRAWINGS

FIG. 1 shows the chemical structure of a segment of a PEG-chitosan molecule wherein the degree of substitution with the PEG group is 0.5.

FIG. 2 shows the chemical structure of a segment of a hyaluronan molecule.

FIG. 3 shows the chemical structure of a segment of the PEG-chitosan of FIG. 1 and a segment of the hyaluronan of FIG. 2 crosslinked by amide linkages between a hyaluronan carboxylate moiety and a PEG-chitosan amino moiety.

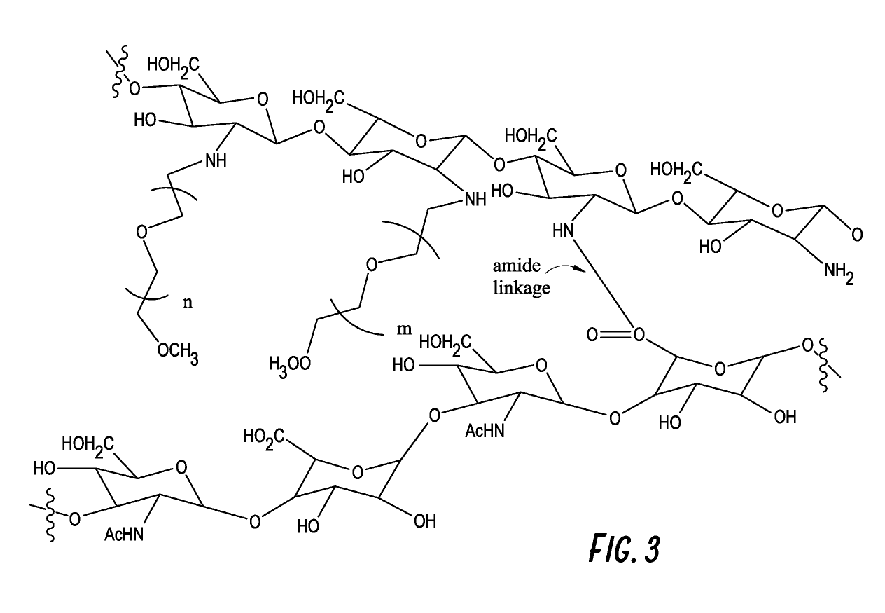

FIG. 4 shows a segment of an acrylated chitosan polymer.

FIG. 5 shows a segment of the acrylated chitosan polymer of FIG. 4 crosslinked by amide linkages formed with a adipic acid.

FIG. 6 shows bevacizumab release from hydrogel depots using (a) real time (PBS pH 7.2) and accelerated conditions (TBS 8.0/8.5) and (b) the effect of pH on acceleration.

FIG. 7 shows OCT images of placebo hydrogel depots (a) and bevacizumab loaded depots (b) showing normal intact retina at 8 weeks.

FIG. 8 shows clearance curves of individual plasma urea levels in Rabbits 1501-1505 after injection.

FIG. 9 shows the preliminary pooled study results of patients treated with 9%, 18% and 36% of intravitreal injection of urea formulations.

FIG. 10 shows the preliminary US study results of patients treated with 9% and 18% of intravitreal injection of urea formulations.

FIG. 11 shows the preliminary ex-US study results of patients treated with 18% and 36% of intravitreal injection of urea formulations.

### DETAILED DESCRIPTION

Embodiments of this disclosure relate generally to the use of stabilized aqueous solutions of urea or urea derivatives for administration to the eyes of humans or other mammals. Urea and urea derivatives liquefy the vitreous humor and induce a posterior vitreous detachment thereby separating the cortical vitreous from the inner limiting lamina of the retina. The strong attachment of the cortical vitreous to the inner limiting lamina of the retina creates traction on the retina which results in rhegmatogenous retinal tears, idiopathic macular holes as well as cystoid macular edema. This separation of the cortical vitreous from the inner limiting lamina of the retina by urea or urea derivatives eliminates the vitreoretinal traction which allows the non-surgical re-attachment of the retinal tear(s), the closure of the macular hole, and the resolution of cystoid macular edema.

In other embodiments, the urea and/or urea derivatives can be incorporated into sustained-release drug delivery systems, including microparticles and implants, such as those reviewed by N. Malavia and P. K. Kaiser, Overview of Sustained-Release Drug-Delivery Systems (March, 2015) Retina Today 67-70 (hereinafter, “Malavia”); and N. Kuno and S. Fujii, Recent Advances in Ocular Drug Delivery Systems (2011) Polymers 3:193-221 (hereinafter, “Kuno”). The contents of both Malavia and Kuno are discussed in more detail below and the contents of both articles are incorporated herein by reference in their entireties.

A. Anatomy of the Human Eye

In human beings, the anatomy of the eye includes a “vitreous body” which occupies approximately four fifths of the cavity of the eyeball, behind the lens. The vitreous body is formed of gelatinous material, known as the vitreous humor. Typically, the vitreous humor of a normal human eye contains approximately 99% water along 30 with 1% macromolecules including: collagen, hyaluronic acid, soluble glycoproteins, sugars and other low molecular weight metabolites.

The retina is essentially a layer of nervous tissue formed on the inner posterior surface of the eyeball. The retina is surrounded by a layer of cells known as the choroid layer. The retina may be divided into a) an optic portion which participates in the visual mechanism, and b) a non-optic portion which does not participate in the visual mechanism. The optic portion of the retina contains the rods and cones, which are the effectual organs of vision. A number of arteries and veins enter the retina at its center, and splay outwardly to provide blood circulation to the retina.

The posterior portion of the vitreous body is in direct contact with the retina. Networks of fibrillar strands extend from the retina and permeate or insert into the vitreous body so as to attach the vitreous body to the retina. (Sebag, J. Graefe's Arch. Clin. Exp. Ophthalmol. 225, 89-93; 1987)

B. The Causes, Treatments and Clinical Sequelae of Rhegmatogenous Retinal Tears, Macular Holes and Cystoid Macular Edema

Diabetic retinopathy, trauma, and other ophthalmological disorders sometimes result in rupture or leakage of retinal blood vessels with resultant bleeding into the vitreous humor of the eye (i.e., “intravitreal hemorrhage”). Such intravitreal hemorrhage typically manifests as clouding or opacification of the vitreous humor.

The human vitreous gel undergoes progressive liquefaction with age. After the age of 40 years, there is a steady increase in observed liquefied vitreous associated with a decrease in vitreous gel volume so that by the age 80 years more than half of the vitreous is liquefied (Mcleod, D, et al. Trans. Ophthal. Soc. UK, 1997; 97:225-231). light microscopic studies of whole human vitreous have demonstrated that vitreous liquefaction initially occurs in pockets, which then coalesce (Sebag. J, et al. Invest. Ophthalmol. Vis. Sci. 1989; 30: 1867-1871). These processes eventually result in rhegmatogenous posterior vitreous detachment (PVD). PVD is usually a sudden event during which liquefied vitreous from the center of the vitreous body bursts through a hole in the posterior vitreous cortex and then dissects the residual cortical gel away from the inner limiting lamina of the retina (Larsson, 1. et al. Graefe'S Arch. Clin. Exp. Opthalmol. 1985; 223: 92-95). The residual vitreous gel then collapses forward to occupy an anterior position in the vitreous cavity. This process may induce a tear in the retina which, in the presence of residual vitreoretinal traction around the break, can result in rhegmatogenous retinal detachment (McLeod, D. et al. Trans. Opthal. Soc. UK 1997; 97: 225-231). Vitreoretinal traction may also result in macular hole formation and it has been suggested that some forms of cystoid macular edema are due to vitreoretinal traction during incomplete PVD (Sebag, J. et al. Survey Opthalmol. 1984; 28: 493-498). In cases where the rhegmatogenous PVD is accompanied by a retinal tear or detachment, it is important that such retinal tear or detachment be promptly diagnosed and surgically repaired. Failure to promptly diagnose and repair the retinal tear or detachment may allow photoreceptor cells of the retina, in the region of the tear or detachment, to die. Such death of the photoreceptor cells of the retina may result in loss of vision. Furthermore, allowing the retinal detachment to remain un-repaired for such extended period of time may result in further intravitreal hemorrhage and/or the formation of fibrous tissue at the site of the hemorrhage. Such formation of fibrous tissue may result in the formation of an undesirable fibrous attachment between the vitreous body and the retina.

The typical surgical procedure used for repair of retinal tears or detachment requires that the surgeon be able to look through the vitreous humor, to visualize the damaged region of the retina (i.e., “transvitreous viewing of the retina”). When intravitreal hemorrhage has occurred, the presence of the hemorrhagic blood within the vitreous can cause the vitreous to become so cloudy that the surgeon is prevented from visualizing the retina through the vitreous. Such hemorrhagic clouding of the vitreous can take −12 months or longer to clear sufficiently to permit trans-vitreal viewing of the retina.

The term Pneumatic Retinopexy was used by Hilton and Grizzard (Hilton, G. F. et al. Ophthalmology 1986; 93: 626-641) as a designation for a nonincisional retinal detachment operation consisting of an intravitreal injection of an expandable gas with cryotherapy and/or photocoagulation of the retinal break(s). Patient positioning oriented the gas bubble to close the retinal break(s), allowing spontaneous resorption of the subretinal fluid. Other authors have reported the complications of subretinal gas (McDonald, H. R. et al. Opthalmology, 1987; 94: 319-326), new retinal break formation (Poliner, L. S. et al. Ophthalmology, 1987; 94: 315-318), macular detachment (Yeo, J. H. et al. Arch. Opthalmol. 1986; 104: 1161-1163) and possible lower success rate in aphakic and pseudophakic eyes (Chen, J. C. et al. Ophthalmology, 1988; 95: 601-608).

Pneumatic Retinopexy is a method of retinal detachment repair which uses cryopexy or photocoagulation in combination with intravitreal gas injection to affect an internal tamponade of retinal breaks. Extension of existing retinal detachments with migration of subretinal fluid into the macula has been reported after pneumatic Retinopexy (Yeo, J. H. et al. Arch. Ophthalmol. 1986; 104: 1161-1163). The present report documents the occurrence of new retinal tears with associated retinal detachment in previously uninvolved quadrants in 20% of the patients within 2-4 weeks of pneumatic Retinopexy (Poliner, L. S. et al. Opthalmology, 1987; 94: 315-318). In these patients the original retinal detachments completely resolved. New retinal tears and associated detachments then developed opposite the original break with vitreous condensation and traction in previously uninvolved quadrants.

The majority of macular holes are “idiopathic” because they occur in eyes that have no previous ocular pathology. Macular holes can form immediately after blunt trauma. Besides trauma, other ocular problems have been associated with macular hole formation, including cystoid macular edema, epiretinal membranes, vitreomacular traction syndrome, rhegmatogenous retinal tears, hypertensive retinopathy, and proliferative diabetic retinopathy (Aaberg, T. M. Survey Opthalmol. 1970; 15: 139-162).

The hallmark complaint of idiopathic macular hole formation is painless central vision distortion or blur of acute or subacute nature. Central visual acuity is initially diminished only mildly; however, as the macular hole progresses over weeks to months, the visual acuity usually deteriorates, then stabilizes around the 20/200 to 20/800 level, and a macular hole diameter of 500 μm.

Examples of substances which have been purported to cause vitreal liquefaction and/or posterior vitreous detachment, or disinsertion are found in the U.S. Pat. No. 4,820,516 (Sawyer), U.S. Pat. No. 5,292,509 (Hageman), and U.S. Pat. No. 5,866,120 (Karageozian et al.).

There exists a need for the elucidation and development of new materials and methods for accelerating the liquefaction of the vitreous and the induction of posterior vitreous detachment, or disinsertion of the vitreous.

### Urea Compositions

In accordance with the present disclosure, urea, a urea derivative, thiourea, a thiourea derivative, guanidine, a guanidine derivative or a compound having General Formula I (below) is administered intravenously to liquefy the vitreous humor and induce a posterior vitreous detachment thereby facilitating separation of the cortical vitreous from the inner limiting lamina of the retina. The strong attachment of the cortical vitreous to the inner limiting lamina of the retina creates traction on the retina, which results in rhegmatogenous retinal tears, idiopathic macular holes as well as cystoid macular edema. This separation of the cortical vitreous from the inner limiting lamina of the retina by urea or urea derivatives eliminates the vitreoretinal traction which allows the non-surgical re-attachment of the retinal tear(s), the closure of the macular hole, and the resolution of cystoid macular edema, among other things. In addition, the occurrence of new retinal tears with associated retinal detachment in pneumatic Retinopexy patients are completely eliminated. The compounds in addition to urea, thiourea, guanidine and derivatives thereof, other compounds useable to carry out the methods of the present disclosure have the General Formula I, as follows:

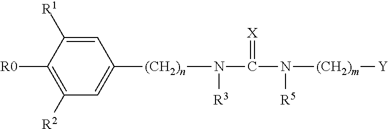

### Wherein:

R is hydrogen, C₁-C₈ alkyl, C₃-C₈ cycloalkyl, C₁-C₆ alkylphenyl or hydroxyl protecting group;

R¹ and R² are each independently hydrogen, C₁-C₆ alkyl, C₃-C₈ cycloalky, C₂-C₆ alkynyl, C₁-C₆ alkoxy, C₂-C₆ alkenyl, C₂-C₆ alkenyl, —S(O)q(C₁-C₆ alkenyl) or

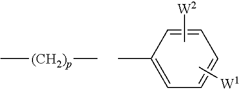

### Wherein:

A is —CH₂—, —O—, —S—, —S(O)— or —S(O)₂—: W¹ and W² are each independently hydrogen, halo, hydroxyl, C₁-C₄ alkyl, C₁-C₄ alkoxy, C₁-C₄ alkylthio, C₂-C₄ alkenyl, or, C₂-C₄ alkynyl;

R³ is hydrogen, C₁-C₈ alkyl, C₈ cycloalkyl, or C₁-C₆ alkylphenyl;

X is 0, S, or NR₄;

R⁴ is hydrogen, C₁-C₆ alkyl, C₁-C₄ alkylphenyl or, C₁-C₆ alkoxy;

R⁵ is hydrogen, C₃-C₈ cycloalkyl or C₁-C₈ alkyl;

Y is

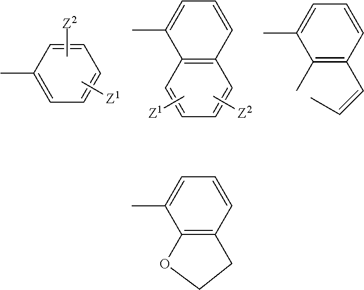

### Wherein:

Z¹ and Z² are each independently hydrogen, C₆ alkyl, C₃-C₈ cycloalkyl, C₁-C₆ alkoxy, hydroxyl, C₂-C₄ alkenyl, C₂-C₆ alkyl, C₁-C₆alkythio, halo, trifluoromethyl or —NR⁶R⁷;

R⁶ and R⁷ are each independently hydrogen, C₃-C₈ cycloalkyl, C₁-C₆ alkyl, C₁-C₄ alkylphenyl;

n is 1 to 6 all inclusive:

m and p are each independently 0 to 6, both inclusive;

q is 0, 1 or 2; and pharmaceutical salts thereof.

Further in accordance with the present disclosure, there are provided methods for treating various ophthalmological disorders in human or veterinary patients by administering to the patient a therapeutically effective amount of urea, a urea derivative, thiourea, a thiourea derivative, guanidine, a guanidine derivative or a compound having General Formula I. The compound may be injected directly into the vitreous body of the eye (Le., intravitreal injection) or may be administered by any other route that causes distribution of a therapeutically effective amount of the compound to the vitreous body. The compound may be administered for the purpose of treating specific ocular diseases or conditions associated with increased intraocular pressure and alterations of the vitreous humor. In one embodiment, the formulations induce a posterior vitreous detachment from the retina thereby separating the cortical vitreous from the inner limiting lamina of the retina. This separation of the cortical vitreous from the inner limiting lamina of the retina by urea or urea derivatives eliminates the vitreoretinal traction which allows the non-surgical re-attachment of the retinal tear(s), the closure of the macular hole, and the resolution of cystoid macular edema, among other things. In addition, the occurrence of new retinal tears with associated retinal detachment in pneumatic Retinopexy patients are completely eliminated. In some embodiments, the compounds can be incorporated into a hydrogel for extended release of the compound into a patient.

Still further in accordance with the present disclosure, there are provided methods for treating rubeosis and/or neovascular glaucoma. Rubeosis is a term that describes abnormal blood vessel growth on the iris and the structures in the front of the eye which are normally devoid of visible blood vessels. Typically, rubeosis results from retinal ischemia in patients who suffer from diabetic retinopathy or vein occlusion. Because the retina is deprived of normal blood flow, the body causes these abnormal blood vessels to form as a compensatory mechanism to supply oxygen to the eye. Unfortunately, the formation of these abnormal vessels often results in obstruction of the angle and/or trabecular meshwork through which aqueous fluid drains from the front of the eye. This causes an elevation in intraocular pressure or “neovascular glaucoma” and a resultant loss of peripheral vision. The present disclosure provides a treatment of rubeosis by administration to the vitreous body of the affected eye an amount of a) urea, a urea derivative, thiourea, a thiourea derivative, guanidine, a guanidine derivative or a compound having General Formula I, b) a nonsteroidal antiinflamatory agent (e.g., ketoralac, diclofenac, etc.) or c) the combination of i) urea, a urea derivative, thiourea, a thiourea derivative, guanidine, a guanidine derivative or a compound having General Formula I and ii) a nonsteroidal antiinflamatory agent (e.g., ketoralac, diclofenac, etc.) sufficient to lessen or prevent the neovascularization of the iris and/or other structures in the front of the eye.

Still further in accordance with the present disclosure, there are provided methods for protecting nerves from damage or for treating damaged nerves to cause regeneration and/or restoration of some impulse transmission through the nerve, by administering or contacting with the affected nerve a therapeutic amount of urea, a urea derivative, thiourea, a thiourea derivative, guanidine, a guanidine derivative or a compound having General Formula I. This method may be used to treat or prevent damage or diminished sensory and/or motor impulse transmission in any nerve, including optic nerves, central nerves (e.g., brain and spinal cord) and peripheral nerves. For this application the compound may be administered by any suitable route including intravitreal injection(to treat or prevent damage to the optic nerve), injection or delivery into the affected nerve, intrathecal, intracranial or intraventricular injection (to treat or prevent damage to central nerves of the brain or spinal cord) or injection or implantation next to or in proximity to the affected nerve.

Still further aspects and elements of the present disclosure will become apparent to those of skill in the art upon reading and understanding of the following detailed description and examples.

One route of administration of the formulations and/or compositions disclosed herein is by intravitreal injection, whereby an aqueous solution of urea, thiourea, guanidine, a derivative of urea, thiourea or guanidine or a compound having General Formula I as shown above is injected directly into the vitreous body located within the posterior chamber of the eye. Alternatively, however, a vitreous liquefying and PVD inducing amount of such compound may be administered by any other suitable route of administration (e.g., topically, etc.) which results in sufficient distribution of the compound(s) to the vitreous body in sufficient amount to cause the desired vitreous liquefaction and posterior vitreous detachment effect. The particular method of administration is not limiting.

The injectable aqueous solution of stabilized urea may contain, certain inactive ingredients which cause the solution to be substantially hypotonic, isotonic, or hypertonic and of a pH range of 4.5-9.0 which is nontoxic for injection into the eye. Such solution of stabilized urea for injection may be in glass vials or pre-filled syringes maintained at room temperature or refrigerated temperature, ready for use. In addition such solutions of stabilized urea may be initially lyophilized to a dry state and thereafter, may be reconstituted prior to use.

Urea employed in the disclosure method can be obtained 99.0-100.0% purity from several manufacturers of fine chemicals.

The present disclosure provides stabilized aqueous urea solutions which comprise a) urea (or a urea derivative), b) sodium chloride, c) citric acid and d) water. One specific example of such a solution is shown in Table I, as follows:

TABLE I
Stabilized Urea Formulation

| Ingredient | Quantity |
|---|---|
| Urea | 0.01%-30.0% by weight |
| Citric Acid | 0.00001%-1.0% by weight |
| Sodium Chloride | 0.05%-3.6% by weight |
| Sterile water for Injection USP | Q.S 100% by weight |

In the stabilized aqueous urea solutions of the present disclosure, the urea concentration is typically in a range of about 0.003 mg per 50 μl to about 50 mg per 50 μl. In one embodiment, urea is provided in the formulation or composition in an amount between 0.005 mg per 50 μl to 7.5 mg per 50 μl. Citric acid is typically provided in a range between about 0.00001% to 1.0%, and in one embodiment, the 25 citric acid is present in an amount between 0.00007% to 0.007%. Sodium chloride is typically provided in the formulation or composition in an amount between 0.05% to 3.6%, and in one embodiment, sodium chloride is provided in an amount between 0.9% and 1.8%. In a preferred embodiment, the composition comprises a combination of urea in an amount between about 0.005 mg per 50 μl to 7.5 mg per 50 μl, between 30 0.00007% and 0.007% citric acid, and between 0.9% to about 1.8% sodium chloride. The formulations disclosed herein may have a pH from about 4.0 to about 9.0, and in a preferred embodiment, the pH is less than 7.0, for example, in a range of 4.0 to 6.5. The formulations may also contain one or more buffers, such as, phosphate buffers, acetate, and/or glycine.

In some embodiments, a pharmaceutical composition for injection into a vitreous body of an eye, comprising a sterile aqueous solution, can comprise
a) 1-99.0% urea by weight;
b) 0.00001-1.0% citric acid by weight;
c) 0.05-3.6% sodium chloride by weight; and
d) sterile water for injection, Q.S. 100% by weight;
   wherein said pharmaceutical composition is stable and non-toxic when injected into the eye.

In some embodiments, the aqueous solution can comprise 6.0-30.0% urea by weight. In some embodiments, the aqueous solution can comprise 15-30.0% urea by weight. In some embodiments, the aqueous solution can comprise 9.0-18.0% urea by weight. In some embodiments, the aqueous solution can comprise 36.0-40.0% urea by weight. In some embodiments, the aqueous solution can comprise 54.0-60.0% urea by weight. In some embodiments, the aqueous solution can comprise urea in a concentration of about 7.5 mg urea per 50 μl. In some embodiments, the aqueous solution can comprise 0.00007-0.007% citric acid by weight. In some embodiments, the aqueous solution can comprise about 0.007% citric acid by weight. In some embodiments, the aqueous solution can comprise 0.9-1.8% sodium chloride by weight. In some embodiments, the aqueous solution can comprise about 0.9% sodium chloride by weight.

In some embodiments, the aqueous solution can have a pH from about 4.0 to about 9.0. In some embodiments, the aqueous solution can have a pH from about 4.0 to about 6.5.

In some embodiments, the pharmaceutical composition can be stable when stored at room temperature for at least about one year. In some embodiments, a dry pharmaceutical composition can be obtainable by lyophilization of the sterile aqueous solution.

In some embodiments, a dry pharmaceutical composition, comprising urea, citric acid and sodium chloride, wherein each of the urea, citric acid and sodium chloride are present in the dry pharmaceutical composition in an amount sufficient to yield an aqueous solution upon reconstitution with water, the aqueous solution can comprise:

a) 1-99.0% urea by weight;

b) 0.00001-1.0% citric acid by weight;

c) 0.05-3.6% sodium chloride by weight; and

d) water, Q.S. 100% by weight; and

wherein after reconstitution of the dry pharmaceutical composition, the aqueous solution is non-toxic when injected into a vitreous body of an eye.

In some embodiments, upon reconstitution with water, the aqueous solution can comprise a urea concentration of 6.0-30.0% by weight. In some embodiments, upon reconstitution with water, the aqueous solution can comprise a urea concentration of 15-30.0% by weight. In some embodiments, the aqueous solution can comprise 9.0-18.0% urea by weight. In some embodiments, the aqueous solution can comprise 36.0-40.0% urea by weight. In some embodiments, the aqueous solution can comprise 54.0-60.0% urea by weight. In some embodiments, upon reconstitution with water, the aqueous solution can comprise a citric acid concentration of 0.00007-0.007% by weight. In some embodiments, upon reconstitution with water, the aqueous solution can comprise a citric acid concentration of about 0.007% by weight. In some embodiments, upon reconstitution with water, the aqueous solution can comprise a sodium chloride concentration of 0.9-1.8% by weight. In some embodiments, upon reconstitution with water, the aqueous solution can comprise a sodium chloride concentration of about 0.9% by weight.

In some embodiments, upon reconstitution with water, the aqueous solution can have a pH from about 4.0 to about 9.0. In some embodiments, upon reconstitution with water, the aqueous solution can have a pH from about 4.0 to about 6.5. In some embodiments, the dry composition can be stable for at least one year at room temperature.

In some embodiments, the composition can be a dry composition comprising:

a) 9-90 mg urea;

b) 0.00003-3 mg citric acid; and

c) 0.15-10.8 mg sodium chloride;

wherein said dry composition is suitable for reconstitution with sterile water for injection to yield an aqueous solution comprising 3-30.0% urea and wherein the aqueous solution is non-toxic when injected into a vitreous body of an eye.

In some embodiments, the composition can comprise 18-90 mg urea. In some embodiments, the composition can comprise 45-90 mg urea. In some embodiments, the composition can comprise 0.00021-0.021 mg citric acid. In some embodiments, the composition can comprise 0.021 mg citric acid. In some embodiments, the composition can comprise 2.7-5.4 mg sodium chloride. In some embodiments, the composition can comprise 2.7 mg sodium chloride.

In some embodiments, the composition can be stable when stored at room temperature for over two years.

In some embodiments, a kit can be used for preparation and/or injection of the aqueous solution shown above. For example, a kit for the preparation of an aqueous solution comprising urea, the aqueous solution being suitable for injection into a vitreous body of an eye, can comprise:

a) a first container containing a dry composition comprising:
i) 9-90 mg urea;
ii) 0.00003-3 mg citric acid; and
iii) 0.15-10.8 mg sodium chloride; and

b) a second container containing at least 300 μL sterile water for injection.

In some embodiments, the kit can further comprise a syringe for intravitreal injection.

In some embodiments, the dry composition can comprise 45-90 mg urea. In some embodiments, the dry composition can comprise 0.021 mg citric acid.

In some embodiments, the first and second containers can be glass vials. In some embodiments, the kit can further comprise instructions for reconstitution and intravitreal injection.

These formulation ingredients are typically initially dissolved in sterile water, sterile filtered and subsequently dispensed as a solution into glass or plastic vials or glass or plastic syringes. In addition the solution could be lyophilized to a dry composition. Thus, the pharmaceutical formulations are liquid formulations, lyophilized formulations, and/or sterile powders. The formulations may be stored in prefilled syringes, in glass vials, and may be stored at room temperature, refrigerated temperature, and frozen temperatures in a sealed condition with little degradation over prolonged periods of time, for example over one year, and in certain embodiments, over three years.

Tables II-IV below show specific examples of stabilized urea preparations that are within the general formulation set forth in the foregoing Table I.

TABLE II

| Ingredient | Quantity |
|---|---|
| Urea USP/NF | 1.50% |
| Citric Acid USP/NF | 0.0007% |
| Sodium Chloride USP/NF | 0.9% |
| Sterile Water for Inj. USP | Q.S 100% |

TABLE III

| Ingredient | Quantity |
|---|---|
| Urea USP/NF | 1.50% |
| Sodium Chloride USP/NF | 0.9% |
| Sterile Water for Inj. USP | Q.S 100% |

TABLE IV

| Ingredient | Quantity |
|---|---|
| Urea USP/NF | 1.50% |
| Sterile Water for Inj. USP | Q.S 100% |

As described in the following examples, the formulations of a stabilized aqueous solution of urea set forth in Table I, II, III and Table IV may be injected directly into the vitreous of the eye at dosage levels which bring about desirable therapeutic effects, including but not necessarily limited to the vitreous liquefaction and PVD induction effect of the present disclosure, without causing significant toxicity to the eye or associated anatomical structures. Among other things, and for example, the amount of urea present in the formulations may be provided in amounts to enhance the therapeutic effects mediated by one or more therapeutic agents. While not wishing to be bound by any particular theory or mechanism of action, it seems that a therapeutic agent that is co-administered with a formulation containing urea is more effectively distributed in the eye to provide a desired therapeutic effect. In other words, coadministration with urea advantageously seems to provide enhanced bioavailability of other therapeutic agents so that the other therapeutic agents can provide one or more therapeutic effects. As used herein, the term “coadministered” does not mean that the urea and the therapeutic agents must be administered precisely at the same time. Instead, in reference to the disclosure herein, coadministration refers to the

TABLE II

| Ingredient | Quantity |
|---|---|
| Urea USP/NF | 1.50% |
| Citric Acid USP/NF | 0.0007% |
| Sodium Chloride USP/NF | 0.9% |
| Sterile Water for Inj. USP | Q.S 100% |

TABLE III

| Ingredient | Quantity |
|---|---|
| Urea USP/NF | 1.50% |
| Sodium Chloride USF/NF | 0.9% |
| Sterile Water for Inj. USP | Q.S 100% |

TABLE IV

| Ingredient | Quantity |
|---|---|
| Urea USP/NF | 1.50% |
| Sterile Water for Inj. USP | Q.S 100% |

As described in the following examples, the formulations of a stabilized aqueous solution of urea set forth in Table I, II, III and Table IV may be injected directly into the vitreous of the eye at dosage levels which bring about desirable therapeutic effects, including but not necessarily limited to the vitreous liquefaction and PVD induction effect of the present disclosure, without causing significant toxicity to the eye or associated anatomical structures. Among other things, and for example, the amount of urea present in the formulations may be provided in amounts to enhance the therapeutic effects mediated by one or more therapeutic agents. While not wishing to be bound by any particular theory or mechanism of action, it seems that a therapeutic agent that is co-administered with a formulation containing urea is more effectively distributed in the eye to provide a desired therapeutic effect. In other words, coadministration with urea advantageously seems to provide enhanced bioavailability of other therapeutic agents so that the other therapeutic agents can provide one or more therapeutic effects. As used herein, the term “coadministered” does not mean that the urea and the therapeutic agents must be administered precisely at the same time. Instead, in reference to the disclosure herein, coadministration refers to the administration of one or more therapeutic agents, other than urea, at a time in which the urea has provided a desired therapeutic effect. Thus, the one or more additional therapeutic agents may be administered in the same formulation comprising urea, or the therapeutic agents may be administered shortly before or after the administration of a formulation containing urea.

In addition, methods of treating neuropathies have also been invented by administering a formulation or composition containing an amount of urea effective to enhance neuronal activity, such as neurotransmission of one or more neurons. Administration of urea-containing formulations results in enhanced optic nerve function in patients who experienced reduced optic nerve function.

### Drug Delivery Hydrogel

In some embodiments, the above described compositions can be incorporated into a hydrogel for use in a patient, such as the hydrogels described below. By incorporating the composition into the hydrogels, the composition can have an extended release when injected into a patient.

Hydrogels are gels in which water is the dispersion medium. A common example of a hydrogel is a gel formed from the protein gelatin in water. Other hydrogels are formed by polysaccharides such as agar dispersed in water. Hydrogels in the form of sheets are used as wound dressings, where they are favored for their ability to help maintain a moist environment to facilitate healing of the wound without drying and cracking of tissues. For example, see www.medicaledu.com/hydrogellsheet.htm. Chemical derivatives of chitosan have also been used to form hydrogels for use as surgical sealants and in drug delivery devices. U.S. Pat. No. 6,602,952, assigned to Shearwater Corp., describes the preparation of poly(alkyleneoxide)chitosan derivatives and their use in the formation of hydrogels. The addition of these hydrophilic but non-ionic groups to the chitosan molecule alters its physical properties. Poly(alkyleneoxides) such as poly(ethyleneoxide), also known (somewhat inaccurately) as poly-ethyleneglycols or PEGs, are formed by the polymerization of alkylene oxides (epoxides) such as ethylene oxide. They may be obtained in a wide variety of molecular weights, with various structural features such as activated end groups, hydrolysable linkages, and others. For example, see the Nektar PEG catalog that lists a wide variety of the Shearwater functionalized PEGs, at www.nektar.com/pdf/nektar_catalog.pdf (as of Aug. 24, 2006).

Chitin, a biopolymer that is abundant in the shells of arthropods, is a β-1,4 polymer of 2-acetamido-2-deoxyglucose. During its isolation, it is freed from proteinaceous and mineral components of the shell. Purified chitin can be further processed by chemical treatment resulting in deacetylation to yield chitosan, (poly-(2-amino-2-deoxyglucose)), which is a basic (alkaline) substance due to its free amino groups. From the perspective of medical uses, chitosan offers several desirable properties. The material is known to be non-toxic and biocompatible, and since chitin is not derived from vertebrates and is processed under rather harsh conditions such as exposure to alkalai during its transformation into chitosan, the possibility of contamination with viruses or prions that are pathogenic to mammals is very low. The utility of biocompatible chitosan derivatives in medical applications has received attention. For example, U.S. Pat. No. 5,093,319 discusses the use of films prepared from carboxymethylated chitosan for use in surgery to prevent post-operative adhesion of injured soft tissues upon healing. The chitosan derivatives are described to be formed into a biodegradable “sheet” that during surgery is emplaced between soft tissues for which adherence during healing is not desired. In another type of use, U.S. Pat. No. 4,532,134 discusses the use of chitosan in promoting blood coagulation in wounds.

Other methods have been described for the preparation of hydrogels from chitosan. The published PCT application WO2005/113608 and the published U.S. patent application no. 2005/0271729, both by the same inventor, discuss the crosslinking of chitosan and hyaluronan, also known as hyaluronic acid. Hyaluronan is an acidic linear polysaccharide formed of β-1,3 linked dimeric units, the dimeric units consisting of an 2-acetamido-2-deoxyglucose and D-gluconic acid linked in a β-1,4 configuration. These published applications discuss crosslinking the two types of polysaccharides using a carbodiimide reagent.

Hydrogels comprising chitosan derivatives and polybasic carboxylic acids or oxidized polysaccharides, for use in vascular occlusion, are also disclosed in copending U.S. patent application Ser. No. 11/425,280, filed Jun. 20, 2006 by the same inventors as in the present application.

As used herein, a “hydrogel” refers to a material of solid or semi-solid texture that comprises water. Hydrogels are formed by a three-dimensional network of molecular structures within which water, among other substances, may be held. The three-dimensional molecular network may be held together by covalent chemical bonds, or by ionic bonds, or by any combination thereof. A common example of a hydrogel is gelatin, a protein, that “sets up” or forms a gel from a sol upon heating and subsequent cooling. Not all substances that form hydrogels are proteins: polysaccharides such as starches may also form hydrogels. Still other hydrogels may be formed through the mixture of two or more materials that undergo chemical reactions with each other to create the three-dimensional molecular network that provides the hydrogel with a degree of dimensional stability. Such mixtures of materials that interact or react with each other to form a hydrogel are referred to herein as a “premix.” Thus, a “premix” as used herein refers to a mixture of materials that after mixing will gel, or “set up,” to form the hydrogel. A premix may be of a liquid or semi-liquid texture such that it can be pumped or transferred by the methods usually used for liquids, such as flow through tubes.

The act of “gelation” refers to the formation of a gel from a sol. In some cases, the sol may consist of a single material dispersed in a solvent, typically water, as in the case of gelatin. In other cases, the sol may consist of more than a single material dispersed in a solvent wherein the several materials will eventually react with each other to form a gel, and when the solvent in which they are dispersed comprises water, the gel is a hydrogel. The hydrogels claimed herein are of the type that are formed by the mixture of more than a single component.

A “saccharide” as used herein refers to a carbohydrate. The term “carbohydrate” includes the class of compounds commonly known as sugars, in addition to compounds that are chemically related to sugars. The term thus includes simple “monosaccharide” sugars, “disaccharide” sugars as well as polymeric “polysaccharides.”. The term encompasses a group of compounds including sugars, starches, gums, cellulose and hemicelluloses. The term further encompasses sugar derivatives such as amino-sugars, for example, 2-amino-2-deoxyglucose, as well as their oligomers and polymers; sulfated sugars; and sugars with hydroxyl, amino, carboxyl and other groups.

A carbohydrate as defined herein comprises sugars or sugar derivatives with beta (β) or alpha (α) anomeric stereochemistry, moreover, the sugars can have (R) or (S) relative configurations, can exist as the (+) or (−) isomer, and can exist in the D or L configuration. The terms “anomer” and “anomeric” refer to the stereochemical configuration at the acetal, hemiacetal, or ketal carbon atom, as is well known in the art.

As used herein, “chitosan” refers to a polysaccharide polymer, either obtained from a natural source such as chitin, or synthetically prepared. Chemically, chitosan is predominantly a polymer of β-1,4-linked 2-amino-2-deoxyglucose monomers. When prepared from a natural source, the usual natural source is chitin, a major constituent of the shells of crabs, shrimp and other arthropods. Chitin is chemically a polymer comprising β-1,4-linked 2-acetamino-2-deoxyglucose monomers. After isolation of chitin from its natural source, it is treated in a manner as to cause hydrolysis of the acetamido group without cleavage of the sugar-sugar bonds, typically through alkaline hydrolysis. Chitosan is not a single molecular entity, but comprises polymeric chains of various lengths.

As used herein, an “alkylated chitosan” is a material formed of chitosan molecules to which carbon-containing molecules have been bonded. The term “alkylated chitosan” thus comprises a large number of possible chemical structures, but they all share the unifying feature that chemical bonds have been formed between the components of the chitosan molecules and at least one carbon atom in each of the molecules that are bonded to the chitosan. For example, methylation of chitosan, in which bonds are formed between methyl radicals or groups and atoms within the chitosan molecule, such as nitrogen, oxygen or carbon atoms, provides an alkylated chitosan within the definition used herein. Other carbon-containing groups may likewise be chemically bonded to chitosan molecules to produce an alkylated chitosan. Specific examples include poly(oxyalkylene)chitosan, wherein poly(oxyethylene), or polyethyleneglycol, chains are covalently bonded to the chitosan backbone, as well as acrylated chitosans, formed by alkylation of chitosan with acrylates.

When referring to the “molecular weight” of a polymeric species such as an alkylated chitosan, a weight-average molecular weight is being referred to herein, as is well known in the art.

A “degree of substitution” of a polymeric species refers to the ratio of the average number of substituent groups, for example an alkyl substituent, per monomeric unit of the polymer as defined.

A “degree of polymerization” of a polymeric species refers to the number of monomeric units in a given polymer molecule, or the average of such numbers for a set of polymer molecules.

A “poly(oxyalkylene)chitosan” is a variety of alkylated chitosan as defined herein. A “poly(oxyalkylene)” group is a polymeric chain of atoms wherein two carbon atoms, an ethylene group, are bonded at either end to oxygen atoms. The carbon atoms of the ethylene group may themselves bear additional radicals. For example, if each ethylene group bears a single methyl group, the resulting poly(oxyalkylene) group is a poly(oxypropylene) group. If the ethylene groups are unsubstituted, the poly(oxyalkylene) group is a poly(oxyethylene) group. A poly(oxyethylene) group may be of a wide range of lengths, or degrees of polymerization, but is of the general molecular formula of the structure [—CH2-CH2-CH2-CH2-O-]n, where n may range from about 3 upwards to 10,000 or more. Commonly referred to as “polyethyleneglycol” or “PEG” derivatives, these polymeric chains are of a hydrophilic, or water-soluble, nature. Thus, a poly(oxyalkylene)chitosan is a chitosan derivative to which poly(oxyalkylene) groups are covalently attached. A terminal carbon atom of the poly(oxyalkylene) group forms a covalent bond with an atom of the chitosan chain, likely a nitrogen atom, although bonds to oxygen or even carbon atoms of the chitosan chain may exist. Poly(oxyethylene)chitosan is often referred to as “polyethyleneglycol-grafted chitosan” or “PEG-chitosan” or “PEG-g-chitosan” or “PEG-grafted-chitosan.”

The end of the poly(oxyethylene) chain that is not bonded to the chitosan backbone may be a free hydroxyl group, or may comprise a capping group such as methyl. Thus, “polyethylene glycol” or “poly(oxyethylene)” or “poly(oxyalkylene)” as used herein includes polymers of this class wherein one, but not both, of the terminal hydroxyl groups is capped, such as with a methyl group. In a specific method of preparation of the poly(oxyethylene)chitosan, use of a polyethyleneglycol capped at one end, such as MPEG (methyl polyethyleneglycol) may be advantageous in that if the PEG is first oxidized to provide a terminal aldehyde group, which is then used to alkylate the chitosan via a reductive amination method, blocking of one end of the PEG assures that no difunctional PEG that may crosslink two independent chitosan chains is present in the alkylation reaction. It is preferred to avoid crosslinking in preparation of the poly(oxyethylene)chitosans of the present disclosure.

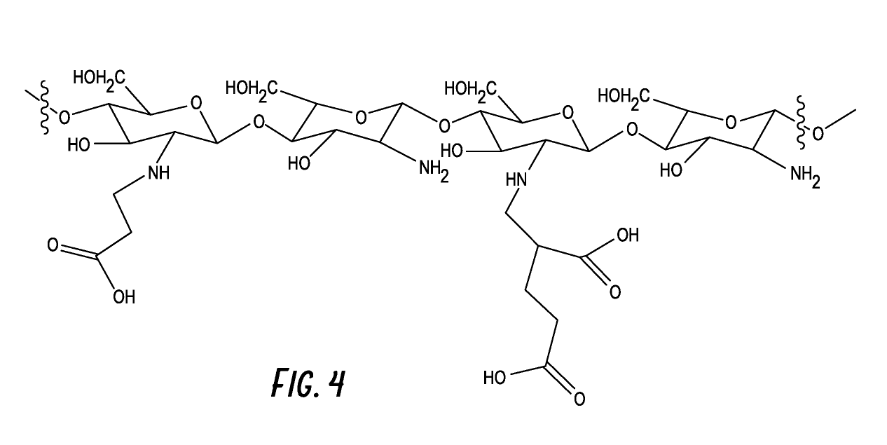

An alkylated chitosan is also a chitosan to which other carbon-containing molecules are linked. An “acrylated chitosan” as the term is used herein is an alkylated chitosan wherein acrylates have been allowed to react with, and form chemical bonds to, the chitosan molecule. An acrylate is a molecule containing an α,β-unsaturated carbonyl group; thus, acrylic acid is prop-2-enoic acid. An acrylated chitosan is a chitosan wherein a reaction with acrylates has taken place. The acrylate may bond to the chitosan through a Michael addition of the chitosan nitrogen atoms with the acrylate. FIG. 4 provides an example of the chemical structure of a segment of an acrylated chitosan polymer.

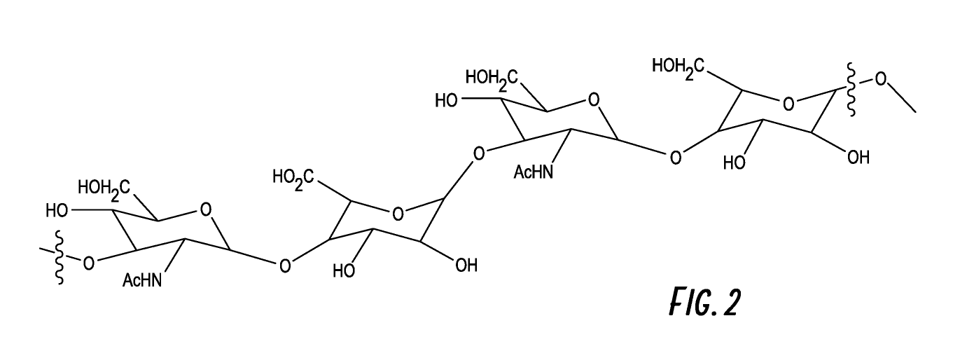

As used herein, a “polybasic carboxylic acid” means a carboxylic acid with more than one ionizable carboxylate residue per molecule. The carboxylic acid may be in an ionized or salt form within the meaning of the term herein. A dibasic carboxylic acid is a polybasic carboxylic acid within the meaning herein. Thus, adipic acid is a polybasic carboxylic acid, having two ionizable carboxylate residues per molecule. Disodium adipate is a polybasic carboxylic acid within the meaning of the term herein. Alternatively, the polybasic carboxylic acid may have hundreds or thousands of ionizable carboxylate groups per molecule; for example, hyaluronan, also known as hyaluronic acid, which is an acidic polysaccharide, is a polybasic carboxylic acid within the meaning assigned herein. The hyaluronan or hyaluronic acid may be in an ionized or salt form within the meaning used herein. Thus sodium hyaluronate is a polybasic carboxylic acid within the meaning of the term as used herein. An example of the chemical structure of a segment of a hyaluronan polymer is shown in FIG. 2.

As used herein, the term “acidic polysaccharide” refers to polymeric carbohydrates comprising carboxylic acid groups. The polymeric carbohydrate can be naturally occurring, or can be synthetic or semi-synthetic. Examples of acidic polysaccharides are hyaluronan and carboxymethyl cellulose. Carboxymethylcellulose, as is well-known in the art, is prepared by reaction of cellulose with sodium chloroacetate, and the product is believed to contain acidic carboxymethyl groups covalently linked to the primary hydroxyl groups of the anhydroglucose monomeric units that make up the cellulose molecule. An oxidized hyaluronan, that is, hyaluronan that has been treated with an oxidizing agent, such as sodium periodate, that cleaves vicinal diol moieties and provides aldehyde groups, is an acidic polysaccharide within the meaning herein, and is also an oxidized polysaccharide within the meaning herein.

The term “oxidized polysaccharide” refers to a polymeric carbohydrate, acidic or non-acidic, that has undergone treatment with an oxidizing reagent, such as sodium periodate, that cleaves vicinal diol moieties of the carbohydrate to yield aldehyde groups. An oxidized hyaluronan, that is, hyaluronan that has been treated with an oxidizing agent, such as sodium periodate, that cleaves vicinal diol moieties and provides aldehyde groups, is an example of an acidic polysaccharide within the meaning herein. An oxidized dextran, that is, dextran that has been treated with an oxidizing agent, such as sodium periodate, that cleaves vicinal diol moieties and provides aldehyde groups, is another example of an oxidized polysaccharide within the meaning herein. Another example of an oxidized polysaccharide is an oxidized starch, that is, a starch that has been treated with an oxidizing agent, such as sodium periodate, that provides aldehyde groups. It is believed that the aldehyde groups of oxidized polysaccharides interact with the amino groups of an alkylated chitosan in such a way as to markedly increase the viscosity of the mixture and cause gelation. While not wishing to be bound by theory, it is believed that this intermolecular interaction takes place through the formation of imines, or Schiff bases, between the amino groups and the aldehyde groups.

A “dehydrating reagent” as used herein refers to a molecular species that takes up the elements of water from a reaction, serving to drive a coupling reaction by thermodynamic factors. A dehydrating reagent is an compound that undergoes reaction of covalent bonds upon taking up the elements of water, as opposed to merely absorbing water into physical particles or the like. Preferably a dehydrating reagent is an organic compound. A specific example of a dehydrating reagent is a carbodiimide, that takes up the elements of water and undergoes changes in covalent bonds to ultimately yield a urea derivative.

As used herein, a “carbodiimide” is a class of organic substances comprising a R—N═C═N—R′ moiety. The R and R′ groups may be any organic radicals. For example, when R and R′ are cyclohexyl groups, the carbodiimide is 1,3-dicyclohexylcarbodiimide, a dehydrating reagent well known in the art. A water-soluble carbodiimide is a carbodiimide that has sufficient solubility in water to form a homogeneous solution at concentrations suitable to carry out the gelation reaction as described herein. Typically, a water-soluble carbodiimide contains an ionic group, such as an ammonium salt, to confer water-solubility upon the molecule. The water-soluble diimide EDCI is 1-ethyl-3-N,N-dimethylaminopropylcarbodiimide.

A “carboxyl activating reagent” as used herein refers to a molecular species that interacts with a carboxyl group in such a way as to render the carbonyl of the carboxyl group more susceptible to nucleophilic attack, as by an amine to yield an amide. This activation may take place by formation of a complex or by formation of a covalent intermediate. A specific example of a carboxyl activating reagent is an N-hydroxy compound that can form an N-hydroxy ester of the carboxylic acid group, increasing the reactivity of the carbonyl moiety to nucleophilic addition of a molecular species such as an amine.

The term “N-hydroxy compound” refers to an organic compound comprising a chemical bond between a hydroxyl group and a nitrogen atom. Specific N-hydroxy compounds such as N-hydroxysuccinimide and N-hydroxybenztriazole (1-hydroxy benzotriazole) are well known in the art as reagents that form esters with carboxylic acid groups and serve to activate the carboxylic acid group in reactions with nucleophiles.

“Gelatin,” as the term is used herein, is a collagen-derived material that is about 98-99% protein by dry weight. The approximate amino acid composition of gelatin is: glycine 21%, proline 12%, hydroxyproline 12%, glutamate 10%, alanine 9%, arginine 8%, aspartate 6%, lysine 4%, serine 4%, leucine 3%, valine 2%, phenylalanine 2%, threonine 2%, isoleucine 1%, hydroxylysine 1%, methionine and histidine <1% and tyrosine <0.5%.

An “aqueous medium,” as the term is used herein, refers to a liquid medium composed largely, but not necessarily exclusively, of water. Other components may also be present, such as salts, co-solvents, buffers, stabilizers, dispersants, colorants and the like.

As used herein, the act of “mixing between mutually coupled syringes” refers to a procedure wherein one syringe is partially filled with one ingredient, a second syringe is partially filled with a second ingredient, and the two syringes are coupled together as with a luer connector such that the contents of the syringes are mixed by drawing the contents of one syringe through the connector into the second syringe, then reciprocally expelling the contents of the second syringe back into the first syringe. This process may be repeated until adequate mixing is achieved.

A “suture” or the act of “suturing” refers to the physical joining of two separate masses of tissue with thread or fiber, or alternatively with solid materials such as fabrics or plastic films on which an adhesive is disposed, whereby the physical joining serves to hold the separate tissue masses in close physical proximity at least temporarily, such as for the period of time required for biological healing to occur. A “suture line” is a line of, for example, stitches of thread as is used to close an incision at the end of a surgical procedure.

A “therapeutic agent” is any agent which serves to repair damage to a living organism to heal the organism, to cure a malcondition, to combat an infection by a microorganism or a virus, to assist the body of the living mammal to return to a healthy state. A “protective agent” is any agent which serves to prevent the occurrence of damage to an organism, such as by preventing the establishment of an infection by a microorganism, to prevent the establishment of a malcondition, to preserve an otherwise healthy body in the state of health. Therapeutic and protective agents comprise pharmaceuticals, radiopharmaceuticals, hormones or their analogs, enzymes, materials for genetic therapy such as antisense nucleotides or their analogs, macroscopic ingredients such as bone powder as is used to induce bone growth, growth factors as may be used to stimulate tissue growth such as by angiogenesis, or any other such agents as are medically advantageous for use to treat a pathological condition. As used herein, “treating” or “treat” includes (i) preventing a pathologic condition from occurring (e.g. prophylaxis); (ii) inhibiting the pathologic condition or arresting its development: (iii) relieving the pathologic condition; and/or (iv) diminishing symptoms associated with the pathologic condition.

A therapeutic agent or a protective agent may comprise a “drug.” As used herein, a “drug” refers to a therapeutic agent or a diagnostic agent and includes any substance, other than food, used in the prevention, diagnosis, alleviation, treatment, or cure of a disease. Stedman's Medical Dictionary, 25th Edition (1990). The drug can include any substance disclosed in at least one of: The Merck Index, 12th Edition (1996); Pei-Show Juo, Concise Dictionary of Biomedicine and Molecular Biology, (1996); U.S. Pharmacopeia Dictionary, 2000 Edition: and Physician's Desk Reference, 2001 Edition. In some embodiments, the therapeutic agent is one of the embodiments of the compositions described above.

A hydrogel for use as a tissue sealant according to the present disclosure is a hydrogel that achieves a gelled state after formation from a premix composed of more than a single component. The hydrogel, which may be used to seal the tissues of a living mammal such as a human patient, is formed upon gelation of the premix, which is in the physical form of a sol. Mixing of the components that make up the premix provides a liquid or semi-liquid sol that may be pumped or transferred by any technique suitable for handling somewhat viscous liquid materials, such as syringes, pipettes, tubing and the like. Upon standing, the premix sol after a period of time sets up into the hydrogel of the present disclosure.

The premix sol and the resulting hydrogel that forms from the sol are suitable for contact with living biological tissue, being biocompatible and preferably biodegradable. Thus, the hydrogel can remain in contact with living biological tissue within a human patient for an extended period of time without damaging the tissue on which it is disposed. In one specific embodiment, the hydrogel has adhesive properties towards living tissues on which it is disposed. In another specific embodiment, the hydrogel contains therapeutic or protective agents that are released into the surrounding tissues on which the hydrogel is disposed. In another specific embodiment, the hydrogel has both adhesive properties towards the tissue on which it is disposed and also contains therapeutic or protective agents that are released into the surrounding tissues on which the hydrogel is disposed. In another specific embodiment the hydrogel contains microparticles or nanoparticles containing therapeutic agents or protective agents that further control the release of the agents from the hydrogel.

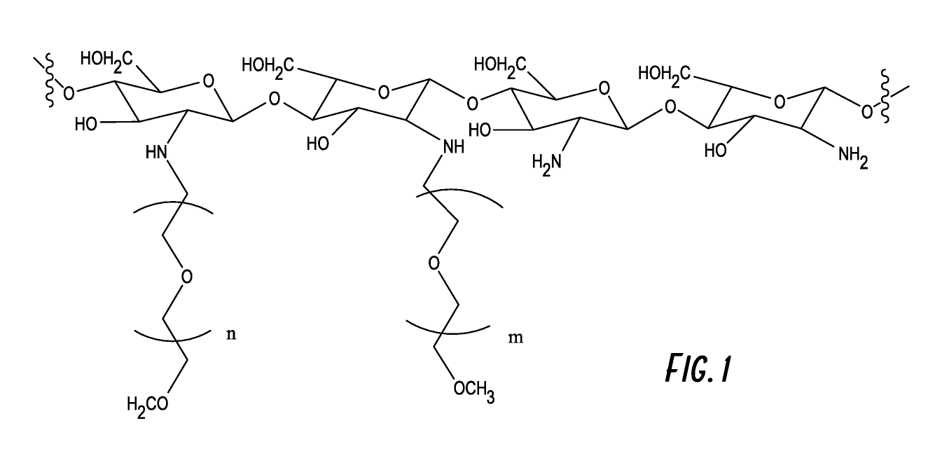

A specific embodiment of a premix that forms a hydrogel according to the present disclosure comprises an alkylated chitosan. Referring to FIG. 1, in a specific embodiment an alkylated chitosan comprises a poly(oxyethylene)chitosan. The poly(oxyethylene)chitosan is a polymer formed of 2-amino-2-deoxyglucose monomeric units. Each monomeric unit comprises a single free amino group and two free hydroxyl groups. In FIG. 1, one amino group is alkylated on the nitrogen atom with a poly(oxyethylene) chain, also known as a polyethyleneglycol chain. In the example provided in FIG. 1, the chitosan has a degree of substitution of 0.5, because two of the four amino groups in the tetrameric unit shown bears the substituent. However, a poly(oxyethylene)chitosan according to the present disclosure may have a degree of amino group substitution ranging down to about 0.1 (wherein only one in about every ten monomeric units is alkylated). Furthermore, a poly(oxyethylene)chitosan according to the present disclosure may also bear the poly(oxyethylene) derivative on one of the two free hydroxyl groups in a given monomeric unit, or may comprises a mixture of N- and O-alkylated chitosan monomeric units, or be di-alkylated or tri-alkylated on a single monomer unit. Thus, a fully alkylated chitosan monomeric unit has a degree of substitution of 3.0, and a poly(oxyethylene)chitosan according to the present disclosure may have a degree of substitution ranging up to 3.0 without departing from the principles of the disclosure.

A preferred degree of substitution for a poly(oxyethylene)chitosan is about 0.35 to about 0.95. A particularly preferred degree of substitution is about 0.5.

It should be understood that other poly(oxyalkylene) groups may be substituted for the poly(oxyethylene) group shown in FIG. 1. For example, a poly(oxypropylene)chitosan may be used in place of, or in addition to, the poly(oxyethylene)chitosan. A poly(oxypropylene) group is the structure that would be obtained if the poly(oxyethylene) group as shown in FIG. 1 bore a methyl group on every ethylene unit (—O—CH2CH(CH3)-O), or alternatively, every ethylene unit shown in FIG. 1 were a 3-carbon linear propylene group (—O—CH2CH2CH2-O—).

The number of monomeric units that make up a chitosan according to the present disclosure may vary widely without departing from the principles of the disclosure. Any sample that contains more than a single molecule of a chitosan derivative will almost inevitably contain a distribution of molecules of different molecular weights. A preferred poly(oxyethylene)chitosan according to the present disclosure has a molecular weight of about 200 kD to about 600 kD.

In a specific embodiment, a premix for a hydrogel contains a polybasic carboxylic acid comprising a hyaluronan. A member of the class of acidic polysaccharides, a hyaluronan bears an ionizable carboxylic acid group on every other monosaccharide residue. The hyaluronan can be in the form of a hyaluronate, that is, with at least most of the carboxylic acid groups being in the ionized or salt form. Sodium hyaluronate is a specific example. Referring to FIG. 2, a hyaluronan or a hyaluronic acid is a polybasic carboxylic acid, and the number of ionizable carboxylate groups per hyaluronan molecule is dependent on the degree of polymerization of the hyaluronan. The degree of substitution of carboxylic acid groups on the polymer backbone, assuming a monomeric unit comprising the disaccharide formed of one glucuronic acid monosaccharide and one 2-acetamido-2-deoxyglucose monosaccharide, is 1.0. Every monomeric unit (disaccharide unit) bears a single ionizable carboxylic acid group. A hyaluronan may be of any of a wide range of degrees of polymerization (molecular weights), but a preferred hyaluronan has a molecular weight of about 2,000 kD to about 3,000 kD. An example of a premix comprising a hyaluronan further comprises a poly(oxyethylene)chitosan, synonymously a PEG-chitosan.

Another specific embodiment of a premix that forms a hydrogel according to the present disclosure comprises an acrylated chitosan. Referring to FIG. 4, in a specific embodiment an alkylated chitosan comprises a acrylated chitosan wherein at least some of the free amino groups of the 2-amino-2-deoxyglycose monosaccharide monomeric units are substituted with acrylate groups. It is believed that acrylate groups are bonded to free amino groups of the chitosan via a Michael type conjugate addition wherein the nucleophilic amino group forms a bond to the β-carbon of the α,β-unsaturated acrylate, but the acrylate may be bonded to the chitosan in a different manner without departing from the principles of the disclosure. Furthermore, as is illustrated in FIG. 4, acrylates may themselves oligomerize after initial alkylation of the chitosan backbone. The three-carbon carboxylic acid substituent on the left illustrates the alkylation of chitosan with a single molecule of acrylate, whereas the six-carbon dicarboxylic acid substituent on the right illustrates the product resulting from addition of a second acrylate molecule to the first acrylate molecule, either prior to or subsequent to addition of the first acrylate molecule to the chitosan backbone.

A preferred degree of substitution of the chitosan backbone with acrylate groups according to the present disclosure is about 0.25 to about 0.45. The number of monomeric units that make up a acrylated chitosan according to the present disclosure may vary widely without departing from the principles of the disclosure. Any sample that contains more than a single molecule of a chitosan derivative will almost inevitably contain a distribution of molecules of different molecular weights. A preferred acrylated chitosan has a molecular weight of about 200 kD to about 600 kD.

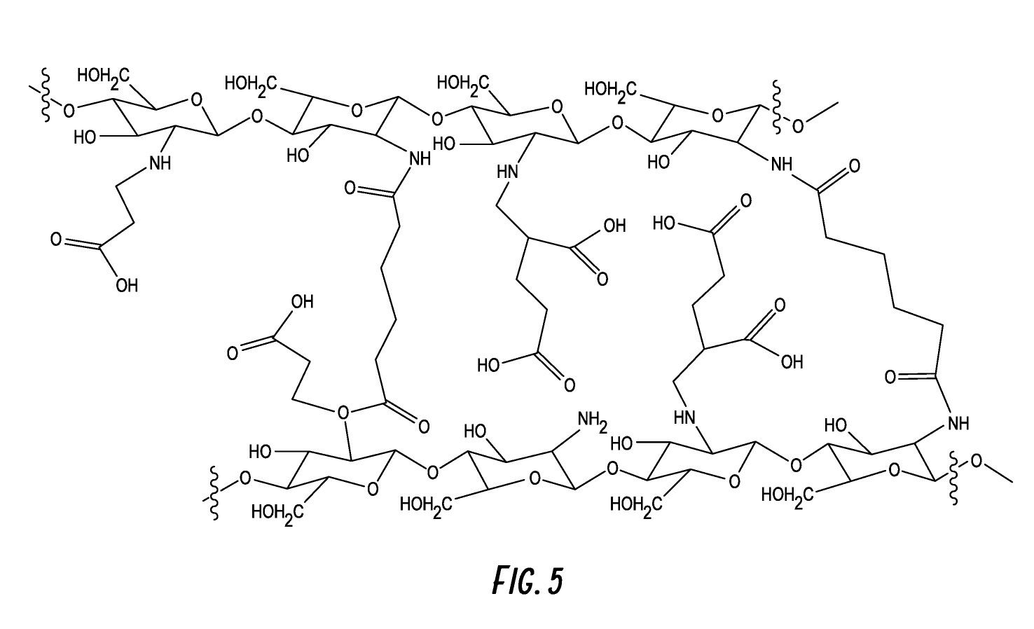

A premix that includes an acrylated chitosan can also include a polybasic carboxylic acid comprising a dicarboxylic acid. A preferred dicarboxylic acid is a dicarboxylic acid wherein the two carboxylate groups are bonded to a moiety of about one to about twelve carbon atoms, which may comprise chains, aliphatic or aromatic rings, or heteroatoms such as nitrogen, oxygen or sulfur. Referring to FIG. 5, a particularly preferred dicarboxylic acid is a linear alkyl α,ω-dicarboxylic acid, which can crosslink acrylated chitosan polymer chains through the intermolecular formation of amide bonds between the chitosan amino groups and the carboxylic acid groups of the dicarboxylic acid. Specific examples of linear alkyl α,ω-dicarboxylic acids are malonic, succinic, glutaric, adipic, pimelic, suberic, azaleic, and sebacic acid. A particularly preferred example is adipic acid. A specific example of a premix of the disclosure comprises acrylated chitosan, disodium adipate, a dehydrating reagent and a carboxyl activating reagent.

In another specific embodiment, a premix that includes an alkylated chitosan also includes a polybasic carboxylic acid comprising a carboxymethylcellulose. A carboxymethylcellulose is a derivative of cellulose (a β-1,4 linked polymer of glucose) wherein hydroxyl groups are substituted with carboxymethyl (—CH2CO2H) moieties. It is understood that the term carboxymethylcellulose comprises salts of carboxymethylcellulose, such as the sodium salt. A specific example of a premix comprises acrylated chitosan, carboxymethylcellulose sodium salt, a dehydrating reagent and a carboxyl activating reagent. Carboxymethylcellulose, as is well-known in the art, may have varying degrees of substitution, a “degree of substitution” referring to the number of derivatizing groups, herein carboxymethyl, per each monomer unit on the average. A particularly preferred carboxymethylcellulose according to the present disclosure has a degree of substitution of about 0.7 and a molecular weight of about 80 kD.

A premix according to the present disclosure comprises an aqueous medium. An aqueous medium necessarily includes water, and may include other components including salts, buffers, co-solvents, additional cross-linking reagents, emulsifiers, dispersants, electrolytes, or the like.

A premix according to the present disclosure can comprise a dehydrating reagent. A preferred dehydrating reagent is a dehydrating reagent that is sufficiently stable when dissolved or dispersed in an aqueous medium to assist in driving the formation of the amide bonds before it is hydrolyzed by the water in the aqueous medium. A particularly preferred type of dehydrating reagent is a carbodxiimide, which is transformed to a urea compound through incorporation of the elements of water. An example of a water-soluble carbodiimide, is 1-ethyl-3-(N,N-dimethylpropyl)carbodiimide (EDCI), which is preferred as it is soluble in the aqueous medium and thus does not require a co-solvent or dispersant to distribute it homogeneously throughout the premix. Other water-soluble carbodiimides are also preferred dehydrating reagents.

A premix according to the present disclosure can comprise a carboxyl activating reagent. A preferred carboxyl activating reagent is a reagent that serves to activate a carboxyl group towards formation of a new bond, such as an amide or ester bond with an amine or a hydroxyl-bearing compound respectively. A specific embodiment of a carboxyl activating reagent reacts with the carboxyl group to form a new compound as an intermediate, which then further reacts with another substance such as an amine to form an amide, or a hydroxyl-bearing compound to form an ester. A preferred carboxyl activating reagent is an N-hydroxy compound. An N-hydroxy compound reacts with a carboxyl group to form an N-hydroxy ester of the carboxylic acid, which may subsequently react with, for example, an amino group to form an amide. An example of an N-hydroxy compound is N-hydroxysuccinimide. Another example of an N-hydroxy compound is N(1)-hydroxybenzotriazole.

Another preferred carboxyl activating reagent is a carbodiimide. A carbodiimide reacts with a carboxyl group to form an O-acylisourea, which may subsequently react with, for example, an amine to form an amide, releasing the carbodiimide transformed through covalent addition of the elements of water to a urea compound. A preferred carbodiimide is a water-soluble carbodiimide, for example EDCI.

In a specific embodiment of the present disclosure, a carbodiimide may serve both as a dehydrating reagent and as a carboxyl activating reagent. Thus, a premix comprising an alkylated chitosan, a polybasic carboxylic acid, and a carbodiimide is a specific embodiment according to the present disclosure. Another specific embodiment is a premix comprising an alkylated chitosan, a polybasic carboxylic acid, a carbodiimide, and another molecular species wherein that species is a carboxyl activating reagent. Another specific embodiment is a premix comprising an alkylated chitosan, a polybasic carboxylic acid, a carbodiimide, and another molecular species wherein that species is a dehydrating reagent.

In an embodiment of a composition of the disclosure, an alkylated chitosan forms a hydrogel after mixing with an oxidized polysaccharide in an aqueous medium. The initial sol formed after mixing undergoes gelation over a period of time, typically a few minutes, to provide a hydrogel of the disclosure. A specific example of an oxidized polysaccharide is oxidized dextran. Additional examples are oxidized starch and oxidized hyaluronan. A specific composition of the disclosure comprises acrylated chitosan and oxidized dextran, which readily forms a hydrogel without addition of a dehydrating reagent or of a carboxyl activating reagent.

Upon standing at room temperature or at the temperature of the human body, a premix sol of the disclosure, after a period of time that is typically in the order of a few minutes, for example about 0.5 minutes to about 20 minutes at about 37° C., or about 1 minute to about 10 minutes at about 37° C., undergoes gelation to form a hydrogel of the disclosure. A premix sol and a resulting hydrogel that forms from the sol are suitable for contact with living biological tissue, being biocompatible and preferably biodegradable. Thus, the hydrogel can remain in contact with living biological tissue within a human patient for an extended period of time without damaging the tissue on which it is disposed.

In an embodiment of the disclosure, the hydrogel contains therapeutic or protective bioactive agents, such as the urea compositions described above, that are released into the surrounding tissues. Bioactive agents can be combined with premix solutions by simply blending commercially available solutions of polypeptides or other agents with the aqueous solutions, with gentle mixing. Cells can likewise be blended with the composition, preferably immediately prior to emplacement to enhance survival of living cells.

In some embodiments, the hydrogel can comprise 1.0-99.0% urea and/or urea derivatives by weight. In some embodiments, the hydrogel can comprise 6.0-60.0% urea and/or urea derivatives by weight. In some embodiments, the hydrogel can comprise 15.0-40.0% urea and/or urea derivatives by weight. In some embodiments, the hydrogel can comprise urea in a concentration that is, is about, is less than, is more than, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, or a range that is between any two of the abovementioned values, urea and/or urea derivatives per 50 μl. In some embodiments, the hydrogel can comprise urea in a concentration of about 7.5 mg urea and/or urea derivatives per 50 μl. In some embodiments, the hydrogel can comprise urea in a concentration of about 9-10 mg urea and/or urea derivatives per 50 μl. In some embodiments, the hydrogel can comprise urea in a concentration of about 18-20 mg urea and/or urea derivatives per 50 μl. In some embodiments, the hydrogel can comprise urea in a concentration of about 27-30 mg urea and/or urea derivatives per 50 μl. In some embodiments, the hydrogel can comprise 0.00007-0.007% citric acid by weight. In some embodiments, the hydrogel can comprise about 0.007% citric acid by weight. In some embodiments, the hydrogel can comprise 0.9-1.8% sodium chloride by weight. In some embodiments, the hydrogel can comprise about 0.9% sodium chloride by weight.

In another embodiment of the disclosure, the hydrogel contains microparticles or nanoparticles containing therapeutic agents or protective bioactive agents such as the urea compositions described above, the microparticles or nanoparticles further controlling the release of the bioactive agents from the hydrogel into the surrounding tissues. A “microparticle” or a “nanoparticle” as used herein is a particulate body of dimensions of the order of microns (micrometers) or nanometers respectively, wherein the particulate body may be hollow or solid. Microparticles and nanoparticles may be formed of organic or inorganic materials. For example, a nanoparticle may comprise a buckminsterfullerene (buckyball), which is organic. Alternatively a nanoparticle may comprise microporous glass, which is inorganic. It is understood that the terms encompass solid lipid nanoparticles, wherein the nanosphere particles are formed from a solid lipid. Preferably the microparticlesor the nanoparticle contains a drug or other substance, the timing of the release of which it is advantageous to control.

Due to the abundance of cationic amino groups in the chitosan structure, it is known that drugs with carboxyl groups can be conjugated thereto and sustained release can be achieved through the hydrolysis of the amide or ester bonds linking drugs to the chitosan molecule. Y. D. Sanzgiri, et al., Pharm. Res., 1, 418 (1990). As a polyelectrolyte, chitosan can also electrostatically conjugate sensitive bioactive agents while preserving their bioactivities and enhancing their stabilities. Such derivatives may be formed with the acrylated chitosan of the present disclosure, and will likewise serve to provide for sustained release and to preserve the bioactivity and to enhance the stability of the conjugated agent(s).

In accordance with other embodiments, the drug used in the therapeutic system will often be placed on, embedded, encapsulated or otherwise incorporated into a delivery matrix. The delivery matrix may be included in or on either the first skeletal structure or the second cushioning structure, or both. The delivery matrix, in turn, comprises either a biodegradable or a non-biodegradable material. The delivery matrix may include, although it is not limited to, a polymer. Examples of biodegradable polymers include protein, hydrogel, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid), and combinations thereof. Non-biodegradable polymers may comprise silicone, acrylates, polyethylenes, polyurethane, polyurethane, hydrogel, polyester (e.g., DACRON@ from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOY® from Elgin Specialty Metals, Elgin, Ill.; CONICHROME® from Carpenter Metals Corp., Wyomissing, Pa.). In one embodiment, the hydrogel may comprise poly(alkyleneoxides), such as poly(ethyleneoxide), also known as polyethyleneglycols or PEGs.

Poly(alkyleneoxides), such as poly(ethyleneoxide), also known as polyethyleneglycols or PEGs, are formed by the polymerization of alkylene oxides (epoxides) such as ethylene oxide. They may be obtained in a wide variety of molecular weights, with various structural features such as activated end groups, hydrolysable linkages, and others. For example, see the Nektar PEG catalog that lists a wide variety of the Shearwater functionalized PEGs, at www.nektar.com/pdf/nektar_catalog.pdf (as of Aug. 24, 2006). PEG hydrogels of varying properties are well known in the art.

To prevent a potential allergic reaction to the ocular insert in a patient, the ocular insert, may comprise a hypoallergenic material. Either or both the first and/or second structure may comprise materials such as hydrogels, polyethylene glycol (PEG), or polyethylene oxide (PEO) that prevent adhesion of proteins and thus minimize the chance of developing an allergic reaction. Alternatively, the drug delivery matrix of the ocular insert may comprise an anti-allergenic and/or antihistaminic compound to prevent an allergic reaction to the ocular insert. In certain embodiments, the delivery matrix may also include other materials known in the art.

### PEG

PEG is a hydrophilic polymer that can be cross-linked to form networks. These networks can incorporate a large water content, allowing the network to swell. Further, due to the high water concentration, PEG can be particularly useful for injection into a vitreous body as it generally elicits a minimal, if any, immune response.

In order to form PEG hydrogels, the ethylene glycol can be cross-linked. This can be done, for example, by using ionized radiation, or through covalent cross-linking of PEG macromers with reactive chain ends. Such PEG macromers with reactive chain ends include acrylate, methacrylate, allyl ether, maleimide, vinyl sulfone, NHS ester, and vinyl ester groups.

The cross-linking mechanism to form hydrogels depends on what the chain ends are of the PEG macromers. Typical cases involve the reaction of vinyl chain ends through the use of a free radical initiator (e.g., ammonium persulfate or TEMED) or light generated radicals. Accordingly, acrylate and methacrylate chain ends undergo polymerization. As the PEG macromers become cross-linked, a PEG hydrogel is formed.

While various methods of gelation (i.e., physical, ionic, or covalent interactions) can be used to form PEG gels, chemically or covalently-crosslinking leads to relatively stable hydrogel structures with tunable physicochemical properties such as permeability, molecular diffusivity, equilibrium water content, elasticity, modulus, and degradation rate

The PEG hydrogels can be especially advantageous for drug release, such as the release of the urea compounds described above. The compositions can be incorporated into the hydrogel and specific degradation rates can be incorporated into the hydrogel. Accordingly, when inserted into a patient, the hydrogel can degrade at a certain rate, thus releasing any chemicals within the hydrogel at approximately that rate. The degradation rate can be relatively fast, such as within minutes, or can be relatively slow, such as hours, days, or months. In some embodiments the compositions can be released through diffusion-controlled, swelling-controlled- and/or chemically-controlled delivery. By adjusting the crosslinking density of the hydrogel, smaller molecules can be released first, and as the hydrogel swells larger and larger molecules can be released.

Further, PEG hydrogels can be reactive to certain external stimuli, such as pH, temperature, and ionic strength, and thus can be designed to release chemicals under certain conditions. However, other methods for chemical release from PEG hydrogels can be used as well.

### Microencapsulation

In some embodiments, the above disclosed urea compositions can be microencapsulated for insertion into a patient. Microencapsulation is the process in which small droplets or particles of liquid or solid material are surrounded or coated by a continuous film of polymeric materials. Firstly the microencapsulation procedure was discovered by Bungen burg de Jon and Kan in 1931 and which were deal with the preparation of gelatin spheres and use of a gelatin coacervation process. The controlled drug delivery system has used to reduce the problems associated with conventional therapy and to improve the therapeutic efficacy of a given drug. The maximum therapeutic efficacy can be achieved by delivering of the active agent in the optimal rate to the target tissue, then causing little toxicity and minimum side effects. To deliver a therapeutic substance to the target site in a sustained controlled release fashion various approaches are used. One is by using microparticles as carriers for drugs. Microparticles are considered as free flowing powders consisting of polymers which are biodegradable in nature and they have the particle size below 200 μm. Microencapsulation process helps for converting the liquids to solids, changing the colloidal and surface properties, providing environmental protection and controlling the release characteristics of different coated materials.

The drug may be as it is or in the form of a pharmacologically acceptable salt thereof (for example, when the drug has a basic group such as amino, salts with inorganic acids such as hydrochloric acid, sulfuric acid and nitric acid, salts with organic acids such as carbonic acid and succinic acid; when the drug has an acidic group such as carboxy, salts with alkali metals such as sodium and potassium, salts with organic bases such as organic amines, e.g. triethylamine, and salts with basic amino acids such as arginine, etc.).

The proper amount of the biologically active substance is dependent on the type of substance, desired pharmacologic effect, duration of action and the like but is generally within the range of about 0.001% to about 90% (w/w) and preferably about 0.01% to about 80% (w/w) based on the shell component biodegradable polymer.

The biodegradable polymer that can be used in the present disclosure is not limited in kind only if it is sparingly soluble or insoluble in water, biocompatible and degradable in the recipient body. The term ‘sparingly soluble’ as used here means a solubility of not more than about 3% (w/v) in water.

The biodegradable polymer which can be used include polymers the weight average molecular weights of which are in the range of about 3,000 to about 30,000, preferably about 5,000 to about 25,000, and more preferably about 5,000 to about 20,000. The dispersity of the biodegradable polymer may range from about 1.2 to about 4.0 and preferably from about 1.5 to about 3.5.

It should be understood that the weight average molecular weight values and the polymer dispersity values mentioned in this specification were determined by gel permeation chromatography (GPC).

The proper amount of said biodegradable polymer is dependent on the strength of pharmacologic activity of the biologically active substance used and the desired rate and duration of release of the same substance. As an example, the biodegradable polymer is used as the microcapsule shell in a proportion of about 0.5 to about 10,000 parts by weight, preferably about 1 to about 100 parts by weight, per part by weight of the biologically active substance.

The preferred biodegradable polymer includes, among others, aliphatic polyesters [e.g. homopolymers (e.g. polylactic acid) or copolymers (e.g. lactic acid/glycolic acid copolymer, 2-hydroxybutyric acid/glycolic copolymer) of .alpha.-hydroxy acids (e.g. glycolic acid, lactic acid, 2-hydroxybutyric acid, 2-hydroxyvaleric acid, 2-hydroxy-3-methylbutyric acid, 2-hydroxycaproic acid, 2-hydroxyisocaproic acid, 2-hydroxycaprylic acid, etc.), cyclic dimers of .alpha.-hydroxy acids (e.g. glycolide, lactide, etc.), hydroxydicarboxylic acids (e.g. malic acid), hydroxytricarboxylic acids (e.g. citric acid), etc., mixtures of such homopolymers and/or copolymers (e.g. a mixture of polylactic acid and 2-hydroxybutyric acid-glycolic acid copolymer)], poly-.alpha.-cyanoacrylic esters, polyamino acids (e.g. poly-.gamma.-benzyl-L-glutamic acid, poly-L-alanine, poly-.gamma.-methyl-L-glutamic acid, etc.), maleic anhydride copolymers (e.g. styrene-maleic acid copolymer) and the like. Preferred, among these, are aliphatic polyesters and poly-.alpha.-cyanoacrylic esters. The most preferred are aliphatic polyesters.

Further preferred, among such aliphatic polyesters, are the homopolymers and copolymers of .alpha.-hydroxy acids or cyclic dimers of .alpha.-hydroxy acids and the mixtures of such homopolymers and/or copolymers. The still more preferred are homopolymers or copolymers of .alpha.-hydroxy acids, and mixtures of the homopolymers and/or copolymers.

Where any of said .alpha.-hydroxy acids, cyclic dimers of .alpha.-hydroxy acids, hydroxycarboxylic acids and hydroxytricarboxylic acids has an optical activity center within its molecule, any of its D-, L- and DL-forms can be employed.

The aliphatic polyester can be easily prepared by the known production technology (cf. JP-A61-28521). The mode of polymerization may be random, block or graft.

The weight average molecular weight of said aliphatic polyester is preferably about 3,000 to about 30,000, more preferably about 5,000 to about 25,000, and most preferably about 5,000 to about 20,000. The dispersity of the aliphatic polyester may range preferably from about 1.2 to about 4.0 and most preferably from about 1.5 to about 3.5.

When the aliphatic polyester is a lactic acid-gly-colic acid copolymer, its copolymerization ratio is preferably about 100/0 through about 50/50 (by weight). When a 2-hydroxybutyric acid-glycolic acid copolymer is used, its copolymerization ratio is preferably about 100/0 through 25/75 (by weight).

The weight average molecular weight of said lactic acid homopolymer, lactic acid-glycolic copolymer or 2-hydroxybutyric acid-glycolic acid copolymer is preferably about 3,000 to about 30,000, most preferably about 5,000 to about 20,000.

When a mixture of lactic acid homopolymer (A) and glycolic acid-2-hydroxybutyric acid copolymer (B) is used as the aliphatic polyester, the (A)/(B) blend ratio is generally about 10/90 through about 90/10 (by weight) and preferably about 25/75 through about 75/25 (by weight).

The weight average molecular weight of lactic acid homopolymer is preferably about 3,000 to about 30,000, most preferably about 5,000 to about 20,000.

The glycolic acid-2-hydroxybutyric acid copolymer is preferably a copolymer consisting of about 40 to about 70 mole % of glycolic acid and the balance of 2-hydroxybutyric acid. The weight average molecular weight of glycolic acid-2-hydroxybutyric acid copolymer is preferably about 5,000 to about 25,000, most preferably about 5,000 to about 20,000.

Regarding the procedures for producing sustained-release microcapsules containing a biologically active substance from a W/O emulsion comprising an inner aqueous phase containing the biologically active substance and an external oil phase containing a biodegradable polymer in accordance with the present disclosure, there can be employed any of the known microencapsulation procedures for biologically active substances, such as the drying-in-water method, coacervation method, spray-drying method, and equivalents thereof.

By way of illustration, the biologically active substance is dissolved in water, to begin with, at the final concentration mentioned above, followed, if necessary, by the dissolution or suspension of a drug retaining substance such as gelatin, agar, alginic acid, polyvinyl alcohol, a basic amino acid or the like to provide an inner aqueous phase.

The inner aqueous phase may further contain a pH control agent for insuring the stability and solubility of the biologically active substance. The pH control agent includes carbonic acid, acetic acid, oxalic acid, citric acid, phosphoric acid, hydrochloric acid, sodium hydroxide, and arginine and lysine as well as salts thereof. The inner aqueous phase may also contain a stabilizer for the biologically active peptide, such as albumin, gelatin, citric acid, sodium ethylenediaminetetraacetate, dextrin, sodium hydrosulfite, polyols such as polyethylene glycol, etc., and/or a preservative such as the conventional p-hydroxybenzoic esters (e.g. methylparaben, propylparaben, etc.), benzyl alcohol, chlorobutanol, thimerosal and so on.

The inner aqueous phase prepared in the above manner is poured into said external (oil) phase containing a biodegradable polymer and the mixture is emulsified to provide a W/O emulsion. This emulsification step can be carried out by any of the conventional dispersing procedures such as intermittent agitation, mixing with a propeller or turbine mixer, colloid mill process, homogenizer process or sonication process.

The above-mentioned biodegradable polymer-containing solution (external oil phase) is a solution of the polymer in an organic solvent. The solvent may be any solvent that boils at a temperature not exceeding about 120° C. and is immiscible with water and capable of dissolving the biodegradable polymer. It can, thus, be selected from among halogenated hydrocarbons (e.g. dichloromethane, chloroform, chloroethanol, dichloromethane, trichloroethane, carbon tetrachloride, etc.), fatty acid esters (e.g. ethyl acetate, butyl acetate, etc.), ethers (e.g. ethyl ether, isopropyl ether, etc.), aromatic hydrocarbons (e.g. benzene, toluene, xylene, etc.) and so on. If necessary, two or more different solvents, among them, can be used in an appropriate ratio.

The resulting W/O emulsion is then subjected to microencapsulation.

For the production of microcapsules from the above W/O emulsion by the drying-in-water method, the W/O emulsion is further added to a third aqueous phase to prepare a ternary W/O/W emulsion and the solvent in the oil phase is then evaporated to provide the desired microcapsules.

An emulsifier may be added to the above external aqueous phase. The emulsifier may generally be any substance that is able to form a stable O/W emulsion. Thus, anionic surfactants (e.g. sodium oleate, sodium stearate, sodium lauryl sulfate, etc.), nonionic surfactants (e.g. polyoxyethylene sorbitan fatty acid esters [Tween 80 and Tween 60, Atlas Powder], polyoxy-ethylene-castor oil derivatives [HCO-60 & HCO-50, Nikko Chemicals], etc., polyvinylpyrrolidone, polyvinyl alcohol, carboxymethylcellulose, lecithin, gelatin, etc. can be mentioned. These emulsifiers can be used alone or in combination. The concentration of the emulsifier can be selected within the range of about 0.01% to about 20%, preferably about 0.05% to about 10%.

Evaporation of the solvent from the oil phase can be carried out by any conventional procedure. Thus, while the system is constantly agitated using a propeller mixer or a magnetic stirrer, the solvent may be evaporated at atmospheric pressure or under gradually decreasing pressure or using a rotary evaporator or the like with the degree of vacuum being controlled as required.

The microcapsules thus formed are collected by centrifugation or filtration, rinsed with distilled water several times to remove the excess biologically active peptide, carrier and emulsifier from the surfaces, then redispersed in distilled water or the like and freeze-dried. To prevent aggregation during the washing procedure, an antiaggregation agent [for example, water-soluble polysaccharides such as mannitol, lactose, glucose, starch (e.g. corn starch), etc., amino acids such as glycine, alanine, etc., proteins such as gelatin, fibrin, collagen, etc. and inorganic salts such as sodium chloride, sodium bromide, potassium carbonate, etc.] may be added. The antiaggregation agent is most preferably mannitol. If necessary, the microcapsules are warmed under reduced pressure to further remove the internal water and organic solvent.

For the production of microcapsules by the coacervation method, a coacervating agent is gradually added to said W/O emulsion with stirring to cause separation and solidification of the high polymer.

The coacervating agent is a polymeric substance or a mineral oil- or vegetable oil-based compound, which is miscible with the solvent for the shell component biodegradable polymer but does not dissolve the biodegradable polymer. Thus, for example, silicone oil, sesame oil, soybean oil, corn oil, cottonseed oil, coconut oil, linseed oil, mineral oil, n-hexane, n-heptane, etc. can be mentioned. If desired, these coacervating agents can be used in combination.

The resulting microcapsules are collected by filtration and washed repeatedly with heptane or the like to remove the coacervating agent. Then, in the same manner as in the drying-in-water method, the excess biologically active substance and solvent are removed.

For the production of microcapsules by the spray drying method, said W/O emulsion is sprayed from the nozzle into the drying chamber of a spray drier so that the organic solvent and water within the finely divided liquid droplets may be evaporated in a brief period of time to provide fine microcapsules. The nozzle mentioned above may for example be a two-fluid nozzle, pressure delivery nozzle, rotary disk nozzle or the like. It is also an effective procedure, if necessary for preventing aggregation of microcapsules, to spray an aqueous solution of said antiaggregation agent from another nozzle concurrently with the spray of said W/O emulsion.

The resulting microcapsules may be warmed under reduced pressure, if necessary, to remove water and the solvent from within the microcapsules.

An antiaggregation agent [for example, water-soluble polysaccharides such as mannitol, lactose, glucose, starch (e.g. corn starch), etc., amino acids such as glycine, alanine, etc., proteins such as gelatin, fibrin, collagen, etc. and inorganic salts such as sodium chloride, sodium bromide, potassium carbonate, etc. and so on] may be added to the microcapsules formed on microencapsulation of a biologically active substance with a biodegradable polymer in the present disclosure The antiaggregation agent is most preferably mannitol.

The particle size of the microcapsules of the disclosure is dependent on the desired rate of delayed release. When the product is intended for injection, the particle size should satisfy the dispersibility and needle passage requirements. Thus, the mean particle diameter may range from about 1 to about 300 μm and preferably from about 5 to about 150 μm.

Of course, there are many methods of encapsulating drugs known to persons having skill in the art, and the type of encapsulation disclosed above is not limiting. All of the known techniques are deemed to be within the scope of the present disclosure.

### Depot Gel Formulations

Small molecule drug formulations according to embodiments of the disclosure can be prepared as depot injections. The environment of use is a fluid environment and may include a subcutaneous, intramuscular, intramyocardial, adventitial, intratumoral, or intracerebral portion, a wound site, or tight joint spaces or body cavity of a human or animal. Multiple or repeated injections may be administered to the subject, for example, when the therapeutic effect of the drug has subsided or the period of time for the drug to have a therapeutic effect has lapsed or when the subject requires further administration of the drug for any reason. The formulation serves as an implanted sustained release drug delivery system after injection into the subject. Such controlled release can be over a period of one week, more than one week, one month, or more than one month. Preferably, the controlled release is over at least a period of one week, more preferably over a period of at least one month.

A small molecule drug formulation according to an embodiment of the disclosure includes a depot gel vehicle. The depot gel vehicle includes a biocompatible polymer, i.e., a polymer that would not cause irritation or necrosis in the environment of use. Biocompatible polymers that may be useful in the disclosure may be bioerodible, i.e., gradually decompose, dissolve, hydrolyze and/or erode in situ. Examples of bioerodible polymers include, but are not limited to, polylactides, polyglycolides, polycaprolactones, polyanhydrides, polyamines, polyurethanes, polyesteramides, polyorthoesters, polydioxanones, polyacetals, polyketals, polycarbonates, polyorthocarbonates, polyphosphazenes, succinates, poly(malic acid), poly(amino acids), polyvinylpyrrolidone, polyethylene glycol, polyhydroxycellulose, polysaccharides, chitin, chitosan, and copolymers, terpolymers and mixtures thereof. The polymer is typically present in the depot gel vehicle in an amount ranging from about 5 to 80% by weight, preferably from about 20 to 70%, often from about 40 to 60% by weight.

In one embodiment, the polymer is a polylactide. A polylactide polymer is a polymer based on lactic acid or a copolymer based on lactic acid and glycolic acid. The polylactide polymer can include small amounts of other comonomers that do not substantially affect the advantageous results that can be achieved in accordance with the disclosure. The term “lactic acid” includes the isomers L-lactic acid, D-lactic acid, DL-lactic acid, and lactide. The term “glycolic acid” includes glycolide. The polymer may have a lactic-acid to glycolic-acid monomer ratio of from about 100:0 to 15:85, preferably from about 60:40 to 75:25, often about 50:50. The polylactide polymer has a number average molecular weight ranging from about 1,000 to about 120,000, preferably from about 5,000 to about 30,000, as determined by gel permeation chromatography. Suitable polylactide polymers are available commercially.

The depot gel vehicle further includes a biocompatible solvent which when combined with the polymer forms a viscous gel, typically exhibiting viscosity in a range from 500 poise to 200,000 poise, preferably from about 1,000 poise to 50,000 poise. The solvent used in the depot gel vehicle is typically an organic solvent and may be a single solvent or a mixture of solvents. To limit water intake by the depot gel vehicle in the environment of use, the solvent, or at least one of the components of the solvent in the case of a multi-component solvent, preferably has limited miscibility with water, e.g., less than 7% by weight, preferably less than 5% by weight, more preferably less than 3% by weight miscibility with water. Examples of suitable solvents include, but are not limited to, benzyl benzoate (BB), benzyl alcohol (BA), ethyl benzoate (EB), triacetin, and N-methyl-2-pyrrolidone (NMP). The solvent is typically present in the depot gel vehicle in an amount ranging from about 20 to 95% by weight, preferably in an amount ranging from about 30 to 80% by weight, often in an amount ranging from about 40 to 60% by weight.

A formulation according to an embodiment of the disclosure includes a small molecule drug dispersed or dissolved in a depot gel vehicle as described above. The term “dispersed or dissolved” is intended to encompass all means of establishing the presence of the small molecule drug in the viscous gel and includes dissolution, dispersion, suspension, and the like. Small molecule drugs used in formulations of the disclosure are sparingly soluble in water. In a preferred embodiment, small molecule drugs used in formulations of the disclosure have less than 1 mg/ml solubility in water. In one embodiment, small molecule drugs used in formulations of the disclosure have a molecular weight in a range from 200 to 2,000 Daltons. Small molecule drugs used in formulations of the disclosure may have a narrow or wide therapeutic window. However, the disclosure generally delivers salubrious results in terms of C_{maχ} and toxicity control for small molecule drugs having a narrow therapeutic window. The small molecule drug is typically present in the formulation in an amount ranging from about 1 to 50% by weight, more preferably in an amount ranging from about 5 to 40% by weight, often in an amount ranging from about 10 to 30% by weight.

### Sustained-Release Drug Delivery Systems

In some embodiments, provided herein are sustained-release drug delivery systems for intravitreal delivery of the urea and/or urea derivatives disclosed herein. The sustained-release drug delivery systems disclosed herein may comprise a variety of biomaterials that are biocompatible, and preferably biodegradable. In some embodiments, the biomaterial may be a polymer, such as a biopolymer. e.g., chitosan, alkylated chitosan, acrylated chitosan, hyaluronan, PEG-Chitosan, or a derivative or combination thereof. In some embodiments, the urea and/or urea derivatives may be encapsulated by the polymer.

The polymer which can be used include polymers the average molecular weights of which are in the range of about 3,000 to about 30,000, preferably about 5,000 to about 25,000, and more preferably about 5,000 to about 20,000. The dispersity of the polymer may range from about 1.2 to about 4.0 and preferably from about 1.5 to about 3.5.

In some embodiments, the polymer and the urea and/or urea derivatives may incorporate into a hydrogel. In some embodiments, the sustained-release drug delivery systems may comprise a depot gel formulation as disclosed herein. In some embodiments, the sustained-release drug delivery systems may comprise a variety of urea and/or urea derivative forms, such as a solution, a hydrogel, a crystalline, a highly dense and concentrated pellet like material, or a combination thereof. In preferred embodiments, the sustained-release drug delivery systems may comprise alternative layers of urea and/or urea derivative, such as solution or crystalline, and hydrogel.

The sustained-release drug delivery systems may comprise a variety of forms, such as implants or injectable particulate systems, e.g., microparticles or nanoparticles, or physical devices, e.g., iontophoresis or microelectromechanical intraocular drug delivery device. Exemplary intravitreal implants include: Durasert™ technology system (pSivida Corp., Watertown Mass.) where the permeability of the polymer layers controls drug release; Retisert® (Bausch & Lomb Inc.), which consists of a central core comprised a silicone elastomer cup with a release orifice that encases a drug tablet coated with a semi-permeable layer of polyvinyl alcohol (PVA) where the size of the elastomer cup's release orifice and the permeability of the PVA layer control the release rate for the drug; Vitrasert® (Bausch & Lomb Inc.), which is an implant comprised of ethylene-vinyl acetate (EVA) and PVA that delivers its drug through an opening in the EVA by passive diffusion; Iluvien® (Alimera Sciences, Alpharetta, Ga.), which is an injectable intravitreal implant made of polyimide and PVA that is injected into the eye for sustained delivery to the back of the eye; Ozurdex® (Allergan, Inc., Irvine, Calif.), is an intravitreal implant comprised of a drug and poly (DL-lactide-co-glycolide) (PLGA) that is injected into the vitreous cavity; I-vation™ (SurModics, Inc. Eden Prairie, Minn.), which is an intravitral helically-coiled titanium implant coated with a drug and non-biodegradable polymers, poly(methyl methacrylate) and EVA; and NT-501 (Neurotech Pharmaceuticals, Inc. Lincoln, R.I.), which is a surgically implantable device that includes a semi-permeable membrane capsule that surrounds a scaffold of strands of polyethylene terephthalate yarn that may include cells; injectable particulate systems, which include: Verisome™ drug delivery platform technology (Icon Bioscience, Inc. Sunnyvale, Calif.), which is a liquid that when mixed with saline, forms a gel that is injected intravitreally; RETAAC system, which is PLGA microspheres with a drug that is injected intravitreously; Cortiject® (NOVA63035, Novagali Pharma S.A.), is an emulsion comprised of an oily carrier with a phospholipid as a surfactant that encapsulates a targeted tissue activated corticosteroid prodrug; and Visudyne® (QLT Ophthalmics, Inc., Menlo Park, Calif.), which is an intravenous liposome formulation; and physical devices, which include: EyeGate II® Delivery System (Eyegate Pharmaceutical, Inc. Waltham, Mass.), which is a system that employs iontophoresis in which a drug is delivered using a weak DC current that pushes charged molecules through the sclera and into the retina, choroid and vitreous; and microelectromechanical systems (MEMS) drug delivery devices, which include (Replenish, Inc. Pasadena, Calif.), have been described in Kuno. More sustained-release systems and formulations are described in WO2012088304, WO2007150018, WO2006017347, WO2005107707, U.S. Pat. No. 8,071,120, EP2033648, EP1742610, CA2545650, CA2406277, etc., the contents of which are hereby expressly incorporated by reference in their entireties.

In preferred embodiments, the intravitreal implant may comprise a canister. In some embodiments, the canister may comprise multiple compartments. A compartment may be open ended to immediately diffuse out the urea and/or urea derivatives as a bolus, or closed ended to limit the diffusion of the urea and/or urea derivatives, e.g., encapsulated in hydrogel or layers thereof.

Preparation processes for implants, microparticles or nanoparticles are known in the art, for example, solvent casting and compression molding, extrusion, emulsion methods, phase separation (coacervation), spray drying, hydrogel template approach (Ohr Pharmaceutical Inc.), as described in Malavia. As further explained in Malavia, Ohr Pharmaceutical's hydrogel template approach uses nanoparticles or microparticles having predefined sizes and shapes with homogeneous size distribution where changes in particle size have a direct impact on the release rates of the drug formulations used with this delivery platform.

Some embodiments further provide methods of using the sustained-release drug delivery systems disclosed herein for treating an eye disorder. For example, the sustained-release drug delivery systems disclosed herein may be used to treat an eye disorder selected from the group consisting of VMA, PVD, diabetic retinopathy, diabetic macular edema, and wet macular degeneration.

In some embodiments, the sustained-release drug delivery systems disclosed herein are administered by intravitreal injection. Either single injection or multiple injections are contemplated, for example, two injections, three injections, four injections, or more. In some embodiments, the sustained-release drug delivery systems may be injected for as many times as needed to achieve a targeted endpoint, such as PVD induction. Injection intervals may vary, for example, the sustained-release drug delivery systems may be injected at each interval that is, is about, is at least, 6 hours, 12 hours, 24 hours, 2 days, 3 days, 4 days, 5 days, 7 days, 14 days, 30 days, 2 months, 3 months, 4 months, 5 months, 6 months, 8 months, 10 months, 12 months, 18 months, 24 months, 36 months, or a time period that is a range between any two of the aforementioned values. In some embodiments, the sustained-release drug delivery systems disclosed herein are administered by intravitreal implant. In some embodiments, the sustained-release drug delivery systems disclosed herein are administered by iontophoresis. In some embodiments, the sustained-release drug delivery systems disclosed herein are administered by a microelectromechanical device. In some embodiments, the sustained-release drug delivery systems disclosed herein are administered in a single dose. In some embodiments, the sustained-release drug delivery systems disclosed herein are administered in multiple doses.

The sustained-release drug delivery systems disclosed herein are capable of extending dwell time of the drug at the vitreoretinal interface to allow the protein denaturant properties to continue. In some embodiments, the sustained-release drug delivery systems may release drug for at least 6 months for a single administration. For example, a single administration of the sustained-release drug delivery system may release drug for a time period that is, is about, is at least, 6 hours, 12 hours, 24 hours, 2 days, 3 days, 4 days, 5 days, 7 days, 14 days, 30 days, 2 months, 3 months, 4 months, 5 months, 6 months, 8 months, 10 months, 12 months, 18 months, 24 months, 36 months, or a time period that is a range between any two of the aforementioned values.

From the foregoing description, it will be appreciated that an inventive urea compositions and methods of use, such as the incorporation of the urea compositions with hydrogels, are disclosed. While several components, techniques and aspects have been described with a certain degree of particularity, it is manifest that many changes can be made in the specific designs, constructions and methodology herein above described without departing from the spirit and scope of this disclosure.

### EXAMPLES

The following examples describe various hydrogel preparations that are compatible with delivery of a therapeutic agent, and in particular, with delivery of urea by intravitreal injection, such as the urea compositions described above.

### Example I

### Treatment of Retinal Tears

Seven male and five female human patients having an average age of 47 years were observed to have single or multiple retinal tears, in addition 9 of the 12 patients had macular detachment. All patients received pneumatic Retinopexy treatment 3 days after intravitreal injection of 1.5 mg of urea in 50 μl of aqueous solution prepared according to the formulation set forth in Table II above. Ophthalmoscopic as well as biomicroscopic examination of all eyes showed no adverse effects of the intravitreal injection of the urea solution. Within 3-7 days after urea injection, 11/12 patients had developed PVD, only 1/12 patients had not developed PVD. Within 90 days of the urea injection that one patient also developed PVD.

Within 3 days of treatment 9/12 patients had complete retinal reattachment and macular attachment, and within 7 days all 12 patients had complete retinal reattachment and macular attachment. All patients were followed up for a period of 90 days, none of the patients experienced an occurrence of new retinal tears with associated retinal detachment.

It has been reported that, only 3% of patients treated by Retinopexy without prior urea treatment improved more than 2 lines of visual acuity (11 letters) at 1 month post Retinopexy and only 7% of the patients improved more than 2 lines of visual 20 acuity (11 letters) at 6 months post Retinopexy. (Tornambe, P. E. et. al. Opthalmology, 1989; 96: 772-783). In this example, 64% of patients who received intravitreal urea injection 3 days prior to pneumatic Retinopexy exhibited an improvement of more than 2 lines of visual acuity (11 letters), and 64% of the patients showed an improvement of more than 2 lines of visual acuity (11 letters) at 6 months post Retinopexy. Also, at 1 month after Retinopexy, the urea-treated patients exhibited an average improvement in visual acuity of 3.9 lines on the eye chart (20 letters) and at 6 months after the Retinopexy, the average improvement of visual acuity per urea-treated patient was 4.2 lines (21 letters). Thus, it is concluded that a) the urea treatment facilitated reattachment of the retinal tears as well as the reattachment of the detached macula of the patients after administration of formulations containing urea, b} as a result of the induction of PVD, the occurrence of new retinal tears with associated retinal detachment does not occur and c) there was improvement in vision in 64% of the Retinopexy patients who received urea treatment compared to only 3%-7% of Retinopexy patients who did not receive urea treatment.

### Example II

### Treatment of Idiopathic Macular Hole

A female patient 58 years old was observed to have a 450 μm idiopathic macular hole of 6 months duration. The macular defect was classified as a stage 3 macular hole, and the patient's visual acuity was recorded at 20/400 at a baseline time point prior to urea treatment. The subject was administered a single intravitreal injection of a formulation containing 1.5 mg of urea in a 50˜1 solution prepared in accordance with the formulation of Table III above, and within 7 days the patient had a complete Posterior Vitreous Detachment (PVD). Ophthalmoscopic as well as Biomicroscopic examination of the patient's eye showed no adverse effects of the intravitreal injection of the urea solution. Seven days after the intravitreal urea injection the patient was administered an intravitreal injection of 0.3 ml of expandable gas (C3 F8).

One week after administration of the gas, the size of the macular hole had decreased from 450 μm to a smaller size of 200 μm and the patient's vision had improved from a baseline visual acuity of 20/400 to 20/80. Two weeks post treatment the size of the macular hole had decreased from 450 μm to a smaller size of 90 μm and the patient's vision had improved from a baseline visual Acuity of 20/400 to 20170. Four weeks post treatment the size of the macular hole had decreased from 450 μm to a smaller size of 60 μm and the patient's vision had improved from a baseline visual acuity of 20/400 to 20/60.

Stage 3 macular holes in patients who have not been administered the formulations disclosed herein do not close by themselves, the conventional way of treating macular holes is to perform a vitreous surgery. The surgery of macular holes has centered around three steps: a) the separation of the posterior hyaloid from the macula and the posterior pole of the eye; b) peeling of the peri hole tissue; and c) use of long acting gas and face-down positioning. The risks of vitreous surgery and the mechanical membrane peeling of the posterior hyaloid by suction may be traumatic to the optic nerve head or the retina itself, leading to retinal hemorrhage, damage to the nerve fiber layer, or even perhaps retinal breaks leading to poor vision.

By administering the PVD-inducing amount of a compound of the present disclosure, the need for vitreous surgery may be completely eliminated. In this manner, the possible complications associated with vitreous surgery and resulting poor vision are avoided. As discussed herein, the compounds of this disclosure liquefy the vitreous humor and induce a posterior vitreous detachment thereby separating the cortical vitreous from the inner limiting lamina of the retina without the need for mechanical disruption of the vitreous. This separation of the cortical vitreous from the inner limiting lamina of the retina eliminates the vitreoretinal traction and thereby allows for nonsurgical re-attachment of the retinal tear(s) and closure of the macular hole.

### Example III

### Intravitreal Urea Injection as an Adjuvant in Pneumatic Retinopexy

The present disclosure provides a method to reduce vitreoretinal traction and induce total posterior vitreous detachment (t-PVD) in subjects with primary regmatogen retinal detachment (PRRD) susceptible of treatment by pneumatic retinopexy was practiced by administering a formulation Containing urea formulation set forth in Table II (VRT-1 001) to the patients.

Consecutive patients of both genders with PRRD eligible for treatment with pneumatic retinopexy where enrolled after informed consent. A 0.3 ml intravitreal injection of the urea formulation set forth in Table II (VRT-1001, Vitreoretinal Technologies Inc., Irvine, Calif., USA) was administered, and pneumatic retinopexy was performed the next day, using 0.4 ml of 100% C3F8. Argon laser was applied as soon as possible to seal the retinal tears. Before, and 1, 7, 15, 30 and 90 days after the procedure, patients were monitored with biomicroscopy, posterior pole photography and retinal fluoroscein angiography (FAG), ultrasound (USG), and electroretinography (ERG). Visual capacity (VC), intraocular pressure (lOP), location of retinal lesions, vitreal status, retinal reapplication, complications, or adverse effects were all recorded. Complete and stable retinal application was considered as a success after a three month follow up.

Twelve eyes of 12 patients with PRRD were assessed. The ages of the patients ranged from between 27 and 60 years (mean 45.8±9.7). 58.3% did not have PVD and 41 0.7% had partial superior PVD by USG. After giving the VRT-1001 injection, all eyes presented t-PVD (75% collapsed and 25% without collapsed). A stable and complete retinal reapplication after the pneumatic retinopexy was achieved in all eyes during the follow up. Final VC was 20/40 or better, in 50% of the eyes (p<0.01). 8.3% maintained their initial VC. 8.3% had 1 line of improvement and 83.4% improved 2 lines. No change in intraocular pressure (lOP) was observed (mean 13.4±3 mmHg). The ERG was sub-normal, as expected after retinal detachments, with no evidence of retinal toxicity, showing an improvement at the end of the study. No alterations were observed in the FAG. Minor complications occurred and they were resolved within a few days. No adverse effects were reported

The methods disclosed herein demonstrate that formulations containing urea are useful as an adjuvant in pneumatic retinopexy to improve the success rate in retinal reapplication.

### Example IV

### Treatment of Vitreous Hemorrhage

The formulations and treatments disclosed herein are also effective to treat vitreous hemorrhage. In this example, seven human patients were treated for diabetes-related nonclearing vitreous hemorrhage. Each patient received a single intravitreal injection of 1001l of a formulation containing urea. The formulation used in this example contained 6% urea, 0.9% sodium chloride, and sterile water for injection (qs 100%).

All seven patients demonstrated clearance of their vitreous hemorrhage sufficient to treat the retina with photocoagulation within 2 to 4 weeks after injection. The rate at which the hemorrhagic blood cleared from the vitreous appears to have been significantly accelerated when compared with normal hemorrhage clearance rates seen in clinical practice.

### Example V

### Treatment as Adjunct to Vitrectomy

The treatment solutions disclosed herein may be injected intravitreally to cause pharmacologic vitreolysis alone or as an adjunct to a vitrectomy procedure.

In this example, five human patients requiring pars plana vitrectomy for various indications were treated one week prior to vitrectomy with a single intravitreal injection of 100 μl of a urea in aqueous solution. The formulation contained 6% urea, 0.9% sodium chloride, and sterile water for injection (qs 100%). All patients underwent routine vitrectomies 8 to 10 days after the urea injection. The minimum waiting time between the urea injection and the vitrectomy procedure is about 1 day to 3 days.

The patients who received the intravitreal injection of urea were noted to have complete vitreous liquefaction and collapse by one week after the injection (i.e., at the time of the virectomy procedure). Unlike patients who do not receive urea injection prior to vitrectomy, the vitreous of urea-treated patients in this example was liquefied to the extent that all or substantially all vitreous traction on the retina was relieved, thereby allowing the surgeon to remove the entire vitreous by aspiration with a syringe system. No vitrectomy cutter was used. Also, in this example, the administration of the urea-containing formulations resulted in a 70% decrease in surgical time relative to the procedures performed on patients who did not receive urea.

### Example VI

### Treatment of Diabetic Retinopathy

An association between a low incidence of progressive retinopathy in subjects with vitreous liquefaction and total PVD (either spontaneous or surgically induced), and a significant risk of aggressive proliferation of new blood vessels in patients with only a partial PVD has been well documented, both in diabetics, (Tagawa, H. et. al. Opthalmology, 1986; 93: 596-601 also Tagawa, H. et. al. Opthalmology, 1986; 93: 1188-1192) as well as in patients with central (Hikichi, T. et. al. RETINA, 1995; 15: 29-33) or branch vein occlusion (Kado, M. et. al. Am J. of Opthalmology, 1988; 105: 20-24).Histopathological observations suggest that the cortical vitreous can provide a scaffold for retinal neovascularization in diabetic retinopathy and other retino-vascular proliferative disorders.

Therefore the ability to prophylactically liquefy the vitreous, collapse the scaffold and induce PVD, could provide an important strategy for diabetic patients in the preproliferative phase to protect them against future retinal or optic disc neovascularization.

Injection of urea into the vitreous results in the breakdown of hyaluronic acid of the vitreous and liquefaction of the vitreous body within several days. In addition, disinsertion of the posterior vitreous has been observed in patients by the use of slit lamp biomicroscopy and in rabbits by specialized histological techniques after urea treatment.

A method of treating and/or preventing diabetic retinopathy includes a step of administering a formulation or composition containing urea to a patient.

Sixty-nine patients with nonproliferative diabetic retinopathy were each administered a single intravitreal injection of 50 UI of a urea formulation containing 3% urea, 0.9% sodium chloride, and sterile water for injection.

All 69 patients exhibited complete vitreous liquefaction and collapse within 2-4 weeks after administration of the formulation. The vitreous liquefaction and vitreous collapse in non-proliferative diabetic retinopathy patients would inhibit the growth and proliferation of the blood vessels, and thus inhibit the progression of diabetic retinopathy. All 69 (of the) patients were followed for six months following the procedure.

### Example VII

### Treatment of Retinitis Pigmentosa

In accordance with the present disclosure, urea, thiourea, guanidine and possibly the other compounds of General Formula above may provide neuroprotective/neuroregenerative effects. In this particular example, patients suffering from retinitis pigmentosa were used to demonstrate the neuroprotective effects.

A double masked placebo controlled, randomized, dose escalation study was done in 32 human patients with retinitis pigmentosa. All patients were treated with a single intravitreal injection of 50 μl of a urea-containing formulation. Three groups of patients received different concentrations of urea. One group received a formulation containing 1.5% urea, 0.9% sodium chloride, and sterile water for injection. A second group received an identical formulation except the formulation contained 3.0% urea. A third group received an identical formulation except the formulation contained 6.0% urea. A placebo group received 50 μl of 0.9% sodium chloride.

Forty-seven percent of the patient reported an increase of at least 3 lines of best corrected visual acuity measured by ETDRS charts, whereas, only 14% of the placebo patients reported an increase of at least 3 lines of best corrected visual acuity measured by ETDRS charts.

The improvement demonstrated by the urea-treated patients corresponds to an average of 10 degrees of improvement in the patient's visual field. In this masked, placebo control study the neuroprotectivel neuroregenerative effects of the urea formulation was demonstrated by a) the increase of visual acuity in 47% of the treated patients b) the corresponding enlargement or increase in the visual fields of these same patients. There was no dramatic increase in the visual field of the placebo control patients.

### Example VIII

### Treatment of Neuropathies

In accordance with the present disclosure, urea, thiourea, guanidine and possibly the other compounds of General Formula I above are effective to treat neuropathies 5 (e.g., disorders or damage to nerves). In this example, 33 non sighted patients with optic nerve damage caused by trauma and/or glaucoma, with visual acuity of light perception or no light perception were administered a urea-containing formulation, as disclosed herein. The study was a double masked placebo controlled, randomized, dose escalation study conducted with 33 human patients with optic nerve damage. Patients were randomized into 5 groups, Group 1 of 5 patients. Group 2 of 4 patients, Group 3 of 8 patients, Group 4 of 8 patients and Group 5 of 8 patients. Each patient received a single intravitreal injection of 50 UI of an aqueous treatment solution, as follows:

| | |
|---|---|
| Group 1 | 0.9% sodium chloride (control) |
| Group 2 | 0.2% by weight Urea |
| Group 3 | 1.5% by weight urea |
| Group 4 | 3.0% by weight Urea |
| Group 5 | 6.0% by weight Urea |

Forty-three percent of the patients exhibited an increase of best corrected visual acuity measured by ETDRS charts. In Six patients the vision improved from No light perception to light perception, in 2 patients the vision improved from light perception to count fingers at 12 inches, in 2 patients the vision improved from no light perception to 20 20/400 and 20/800 vision and in 2 patients the vision improved from light perception to 20/600 and 20/800 vision. No patients in the placebo group reported any improvement in best corrected visual acuity as measured by ETDRS charts.

In another study, three patients with long standing (6 months or longer) optic nerve damage related to optic nerve tumors were treated with a single intravitreal injection of 100 μl of a 6% urea containing formulation. All three patient treated reported a significant improvement in visual acuity after treatment. The patients' visual acuity improved from an average of Count Fingers at 12 inches to 20/200.

### Example IX

### Treatment of Rubeosis

In accordance with the present disclosure, urea, thiourea, guanidine and possibly the other compounds of General Formula I alone and/or in combination with nonsteroidal anti-inflammatory agents (e.g., ketoralac, diclofenac, flourbiprofen, Ibuprofen etc.) may be effective in treating rubeosis. In this example, a single patient suffering from rubeosis received a single intravitreal injection of 100 μl of an aqueous solution containing 6.0% by weight urea, 0.9% by weight sodium chloride, and 0.2% by weight ketoralac in a (and) sterile water for injection (qs 100%). The patient was followed for 10 weeks.

The patient was noted to have a dramatic reduction in neovascularization of the iris as documented by iris fluoroscein angiography before and after the urealketorolac injection.

### Example X—Treatment of Diabetic Macular Edema

In accordance with the present disclosure, urea, thiourea, guanidine and possibly the other compounds of General Formula I above may be administered alone and/or in combination with a nonsteroidal anti-inflammatory agent (e.g., ketoralac, diclofenac, flourbiprofen, ibuprofen, etc.) to treat macular edema. In this example, a patient with macular edema related to diabetic retinopathy received a single intravitreal injection of 100l of an aqueous solution containing 6.0% by weight urea, 0.9% by weight sodium chloride and 0.07% by weight ketoralac in sterile water for injection (qs 100%). The patient was noted to have a dramatic improvement in best corrected visual acuity of at 25 least 3 lines by ETDRS measurements during the course of the 8 week monitoring cycle.

### Example XI—Preparation of Acrylated Chitosan

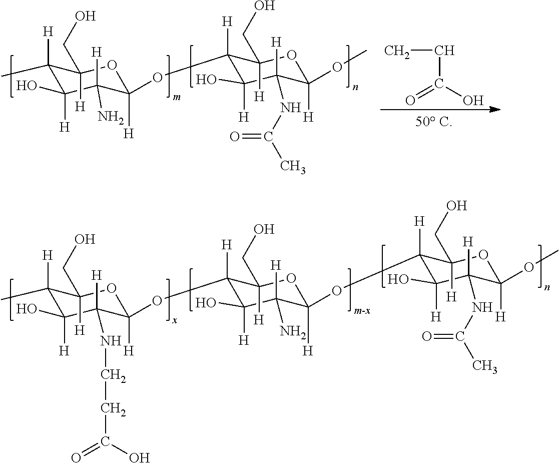

5.52 ml of acrylic acid was dissolved in 150 ml of double distilled water and 3 g of chitosan (Kraeber® 9012-76-4, molecular weight 200-600 kD) was added to it. The mixture was heated to 50 C and vigorously stirred for 3 days. After removal of insoluble fragments by centrifugation, the product was collected and its pH was adjusted to 11 by adding NaOH solution. The mixture was dialyzed extensively to remove impurities.

### Example XII—Preparation of PEG-Chitosan

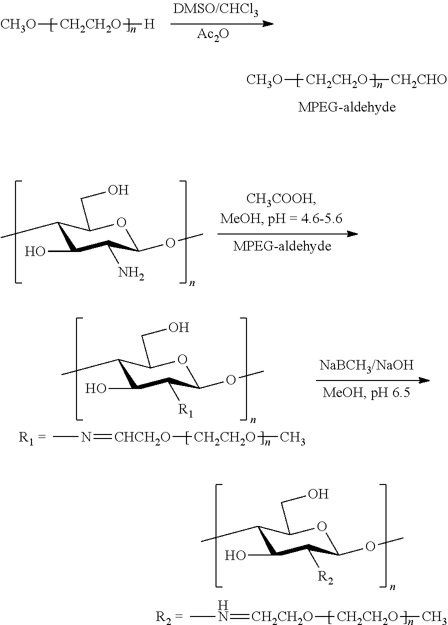

Monomethyl-PEG-aldehyde was prepared by the oxidation of Monomethyl-PEG (MPEG) with DMSO/acetic anhydride: 10 g of the dried MPEG was dissolved in anhydrous DMSO (30 ml) and chloroform (2 ml). Acetic anhydride (5 ml) was introduced into the solution and the mixture is stirred for 9 h at room temperature. The product was precipitated in 500 ml ethyl ether and filtered. Then the product was dissolved in chloroform and re-precipitated in ethyl ether twice and dried.

Chitosan (0.5 g, 3 mmol as monosaccharide residue containing 2.5 mmol amino groups, Kraeber 9012-76-4, molecular weight 200-600 kD) was dissolved in 2% aqueous acetic acid solution (20 ml) and methanol (10 ml). A 15 ml sample of MPEG-

The patient was noted to have a dramatic reduction in neovascularization of the iris as documented by iris fluoroscein angiography before and after the urea/ketorolac injection.

### Example X

### Treatment of Diabetic Macular Edema

In accordance with the present disclosure, urea, thiourea, guanidine and possibly the other compounds of General Formula I above may be administered alone and/or in combination with a nonsteroidal anti-inflammatory agent (e.g., ketoralac, diclofenac, flourbiprofen, ibuprofen, etc.) to treat macular edema. In this example, a patient with macular edema related to diabetic retinopathy received a single intravitreal injection of 100 μl of an aqueous solution containing 6.0% by weight urea, 0.9% by weight sodium chloride and 0.07% by weight ketoralac in sterile water for injection (qs 100%). The patient was noted to have a dramatic improvement in best corrected visual acuity of at 25 least 3 lines by ETDRS measurements during the course of the 8 week monitoring cycle.

### Example XI

### Preparation of Acrylated Chitosan

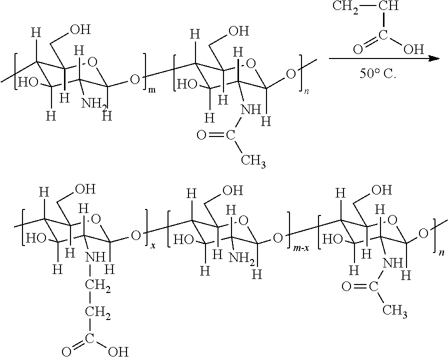

5.52 ml of acrylic acid was dissolved in 150 ml of double distilled water and 3 g of chitosan (Kraeber® 9012-76-4, molecular weight 200-600 kD) was added to it. The mixture was heated to 50 C and vigorously stirred for 3 days. After removal of insoluble fragments by centrifugation, the product was collected and its pH was adjusted to 11 by adding NaOH solution. The mixture was dialyzed extensively to remove impurities.

### Example XII

### Preparation of PEG-Chitosan

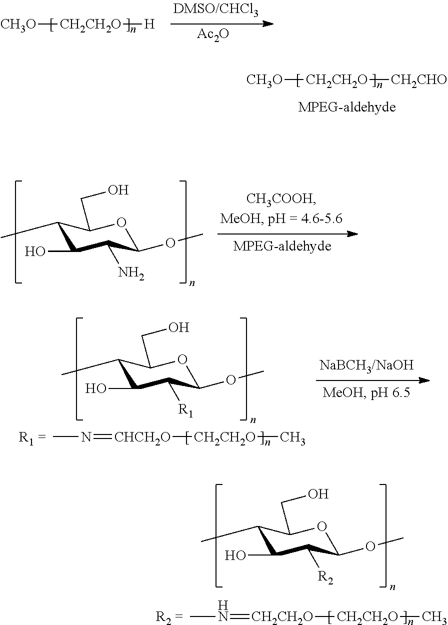

Monomethyl-PEG-aldehyde was prepared by the oxidation of Monomethyl-PEG (MPEG) with DMSO/acetic anhydride: 10 g of the dried MPEG was dissolved in anhydrous DMSO (30 ml) and chloroform (2 ml). Acetic anhydride (5 ml) was introduced into the solution and the mixture is stirred for 9 h at room temperature. The product was precipitated in 500 ml ethyl ether and filtered. Then the product was dissolved in chloroform and re-precipitated in ethyl ether twice and dried.

Chitosan (0.5 g, 3 mmol as monosaccharide residue containing 2.5 mmol amino groups, Kraeber 9012-76-4, molecular weight 200-600 kD) was dissolved in 2% aqueous acetic acid solution (20 ml) and methanol (10 ml). A 15 ml sample of MPEG-aldehyde (8 g, DC: 0.40) in aqueous solution was added into the chitosan solution and stirred for 1 h at room temperature. Then the pH of chitosan/MPEG-monoaldehyde solution was adjusted to 6.0-6.5 with aqueous 1 M NaOH solution and stirred for 2 h at room temperature. NaCNBH3 (0.476 g, 7.6 mmol) in 7 ml water was added to the reaction mixture dropwise and the solution was stirred for 18 h at room temperature. The mixture was dialyzed with dialysis membrane (COMW 6000-8000) against aqueous 0.5 M NaOH solution and water alternately. When the pH of outer solution reached 7.5, the inner solution was centrifuged at 5,000 rpm for 20 min. The precipitate was removed. The supernatant was freeze-dried and washed with 100 ml acetone to get rid of unreacted MPEG. After vacuum drying, the final product (white powder) was obtained as water soluble or organic solvent soluble PEG-g-Chitosan. The yield of water soluble derivatives was around 90% based on the weight of starting chitosan and PEG-aldehyde.

### Example XII

### Preparation of a PEG-Chitosan and Hyaluronan Hydrogel

Hyaluronan (sodium hyaluronate, Kraeber 9067-32-7) was dissolved in water as a 0.5% solution by weight. PEG-chitosan, prepared as described in Example 2, was dissolved in water as a 5% solution by weight. A sample of each solution (0.5 mL of each) was mixed, then a solution of EDCI (20 μL of a solution in water at 350 mg/mL) was added and the solution was thoroughly mixed. Immediately a solution of N-hydroxysuccinimide (20 μL of a solution in water at 125 mg/mL) was added and thoroughly mixed in to form a premix. The premix gelled into a hydrogel in about 7 minutes at ambient temperature (22° C.). At 37° C. gelation occurred in about 2 minutes.

### Example XIV

### Preparation of an Acrylated Chitosan and Adipic Acid Hydrogel

A sample of acrylated chitosan prepared as described in Example XI was dissolved in water at a concentration of 2% by weight. A sample of this solution (0.5 mL) was mixed with a solution of adipic acid in water (40 μL of a 20 mg/mL solution), then a solution of EDCI (20 μL of a 350 mg/mL solution) and the solution thoroughly mixed. Then, a solution of N-hydroxysuccinimide in water (20 μL of a 125 mg/mL solution) was mixed in. The premix gelled in about 9 minutes at ambient temperature (22° C.). At 37° C. gelation occurred in about 3 minutes.

### Example XV

### Preparation of an Acrylated Chitosan and Carboxymethylcellulose Hydrogel

A sample of acrylated chitosan prepared as described in Example XI was dissolved in water at a concentration of 2% by weight. A sample of carboxymethylcellulose sodium salt (Polysciences no. 06140, MW 80 kD, degree of substitution 0.7) was dissolved in water at a concentration of 5% by weight. These two solutions (0.25 mL each) were mixed with a solution of EDCI (20 μL of a 6.5% solution) and the solution thoroughly mixed. Then, a solution of N-hydroxysuccinimide in water (20 μL of a 35% solution) was mixed in. The solution gelled in about 10 minutes at ambient temperature (22° C.).

### Example XVI

### Preparation of Oxidized Dextran

Dextran (5 g) was dissolved in 400 mL of distilled H2O, then 3.28 g of NaIO4 dissolved in 100 mL ddH2O was added. The mixture was stirred at 25° C. for 24 hrs. 10 ml of ethylene glycol was added to neutralize the unreacted periodate following by stirring at room temperature for an additional hour. The final product was dialyzed exhaustively for 3 days against doubly distilled H2O, then lyophilized to obtain a sample of pure oxidized dextran.

### Example XVII

### Analyses of Oxidized Dextran

The degree of oxidation of the oxidized dextran was determined by quantifying the aldehyde groups formed using t-butyl carbazate titration via carbazone formation. A solution of oxidized dextran (10 mg/ml in pH 5.2 acetate buffer) was prepared; and a 5-fold excess tert-butyl carbazate in the same buffer was added and allowed to react for 24 hrs at ambient temperature, then a 5-fold excess of NaBH3CN was added. After 12 hrs, the reaction product was precipitated three times with acetone and the final precipitate was dialyzed thoroughly against water, followed by lyophilization. The degree of oxidation (i.e., abundance of aldehyde groups) was assessed using 1H NMR by integrating the peaks: 7.9 ppm (proton attached to tert-butyl) and 4.9 ppm (anomeric proton of dextran).

### Example XVIII

### Preparation of an Oxidized Dextran/Acrylated Chitosan Gel

A 1 mL sample of a 1-3% aqueous oxidized dextran in water solution was mixed with 1 mL of a 1-3% aqueous acrylated chitosan solution. The mixture was gently stirred for 10 seconds. Gelation occurred within 30 seconds to 10 minutes at temperatures ranging from 5° C. to 370° C.

### Example XIX

### Preparation of Oxidized Hyaluronan

Sodium hyaluronate (1.0 gram) was dissolved in 80 ml of water in a flask shaded by aluminum foil, and sodium periodate (various amounts) dissolved in 20 ml water was added dropwise to obtain oxidized hyaluronan (oHA) with different oxidation degrees. The reaction mixture was incubated at ambient temperature and 10 ml of ethylene glycol was added to neutralize the unreacted periodate following by stirring at room temperature for an additional hour. The solution containing the oxidized hyaluronan was dialyzed exhaustively for 3 days against water, then lyophilized to obtain pure product (yield: 50-67%).

### Example XX

### Analyses of Oxidized Hyaluronan

The degree of oxidation of oxidized hyaluronan was determined by quantifying aldehyde groups formed with t-butyl carbazate titration via carbazone formation. A solution of the oxidized hyaluronan (10 mg/ml in pH 5.2 acetate buffer) and a 5-fold excess tertbutyl carbazate in the same buffer were allowed to react for 24 hrs at ambient temperature, followed by the addition of a 5-fold excess of NaBH3CN. After 12 hrs, the reaction product was precipitated three times with acetone and the final precipitate was dialyzed thoroughly against water, followed by lyophilization. The degree of oxidation (i.e., abundance of aldehyde groups) was assessed using 1H NMR by integrating the peaks: 1.32 ppm (tert-butyl) and 1.9 ppm (CH3 of hyaluronic acid).

### Example XXI

### Preparation of a PEG-Chitosan/Hyaluronan Hydrogels

A solution of PEG-chitosan (2.5 wt %) and hyaluronan (0.5%) in water at pH in the range of about 3.5 to about 5.5 was made up by first making solutions of the two polymers independently at the stated pH, then mixing the solutions. The mixture rapidly formed a viscous solution.

A solution of PEG-chitosan (1.3% w/v, 25 g), hyaluronan (2% w/v, 2.3 g), and 0.1 M HCl (pH 1.45, 0.45 g) was made up. The viscosity increased markedly within about 30 seconds. The pH of the resulting mixture was within the 3.5 to 5.5 range.

### Example XXII

### Preparation of a Gelatin/Oxidized Hyaluronan Hydrogel

A 20% w/v solution of gelatin in water (1 ml) was mixed with a 20% solution of partially oxidized Hyaluronan (1 ml) (20.3% oxidized). The solution was warmed to about 40-450° C., above the melting point of the gelatin, and was mixed. At about 37° C. gelation occurs in about 15 minutes. Optionally, a buffer system of 0.1 M borax at pH 9.4 may be used.

### Example XXIII

### Preparation of an Extended Release Urea Hydrogel

A pharmaceutical composition for injection into a vitreous body of an eye, is made by mixing a sterile aqueous solution, comprising: a) 3-30.0% urea by weight; b) 0.00001-1.0% citric acid by weight; c) 0.05-3.6% sodium chloride by weight; and sterile water for injection, Q.S. 100%, with a 20% w/v solution of gelatin in water (1 ml) mixed with a 20% solution of partially oxidized Hyaluronan (1 ml) (20.3% oxidized). The aqueous solution is warmed to about 40-45° C., above the melting point of the gelatin, and mixed.

### Example XXIV

### Preparation of an Extended Release Urea in a PEG Hydrogel

A pharmaceutical composition for injection into a vitreous body of an eye, is made by mixing a sterile aqueous solution, comprising: a) 3-30.0% urea by weight; b)hydrogel. The PEG hydrogel can be formed either prior to injection, or after injection in a patients vitreous body.

### Example XXV

### Treatment of Eye Disorders with Extended Release Urea in a PEG Hydrogel

Non-diabetic patients with symptomatic VMA (vitreomacular adhesion) are injected with a PEG hydrogel comprising 50%% urea in vehicle (disclosed above in Example XXIII) is injected intravitreally. Patients are monitored daily for 60 days. Control patients receive vehicle only in hydrogel. Resolution rates of more than 50% may be seen after 30 days and total PVD in over 30% of patients after 60-90 days.

### Example XXVI

### Extended Release Testing

As discussed above, injections of urea based compounds have been shown to manage the progression of certain eye diseases to prevent further vision loss and, in some case, gain visual acuity. However, frequent intravitreal injections can increase the risk of infection, retinal detachment, and/or hemorrhage.

Accordingly, as disclosed above, an extended or sustained release methodology has been developed through the use of hydrogels. Testing was done to evaluate the sustained release of bevacizumab from preformed hydrogel depots for intravitreal injections.

In the testing, fine particles of bevacizumab were formulated in PEG hydrogels to form injectable depots. Then, the depots were examined in vitro for sustained release under accelerated conditions in TBS (pH 8.0 and 8.5) and real-time conditions in PBS (pH 7.2) at 37° C. Accelerated and real-time in vitro release was explored to determine release profiles and aggregation (by SEC) as a result of the gradual hydrogel degradation. In vivo assessment was conducted in rabbits (8 eyes) using 10 μl depot for up to 8 weeks using ocular clinical observations and Optical Coherence Tomography (OCT).

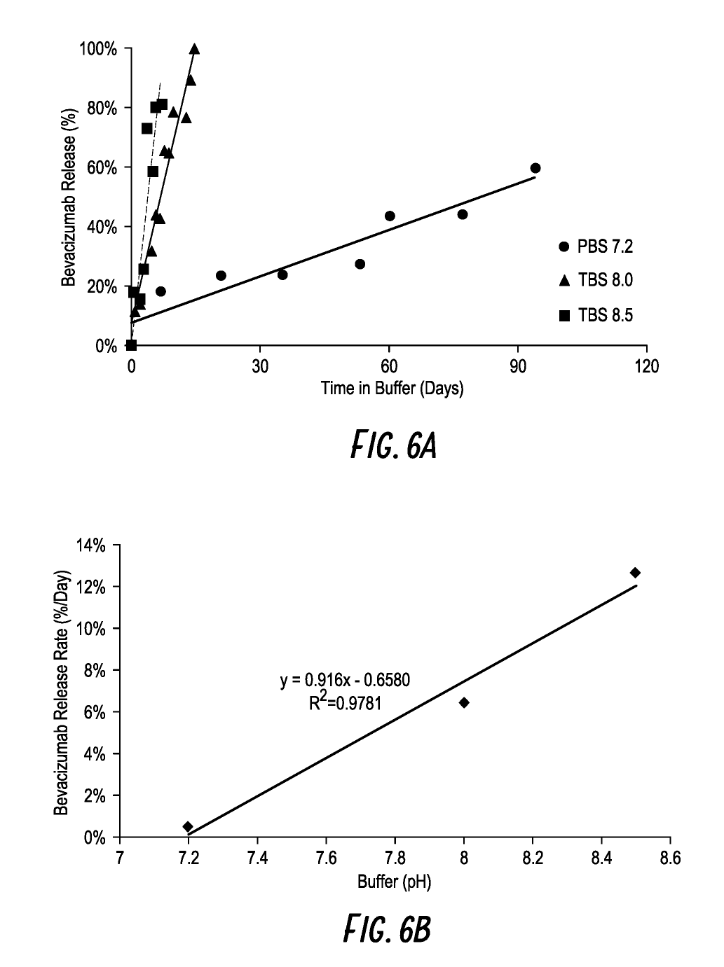

FIG. 6 illustrates release profiles showing the effect of hydrogel degradation time under various pH conditions on sustained release of bevacizumab. A real-time in vitro release profile (PBS 7.2) shoed a controlled low burst (10%) and release up to 3 months, with total projected release at 5-6 months. Accelerated release kinetics showed a 24x acceleration factor (at pH 8.5) and 12x (at pH 8.0), when compared to real-time release. High total percent recover (>90%) and good correlation of release rate to pH were also concluded, as shown in FIG. 6, indicating drug release correlation with gel hydrolysis. Further, minimal high molecular weight species were observed, indicating low aggregation.

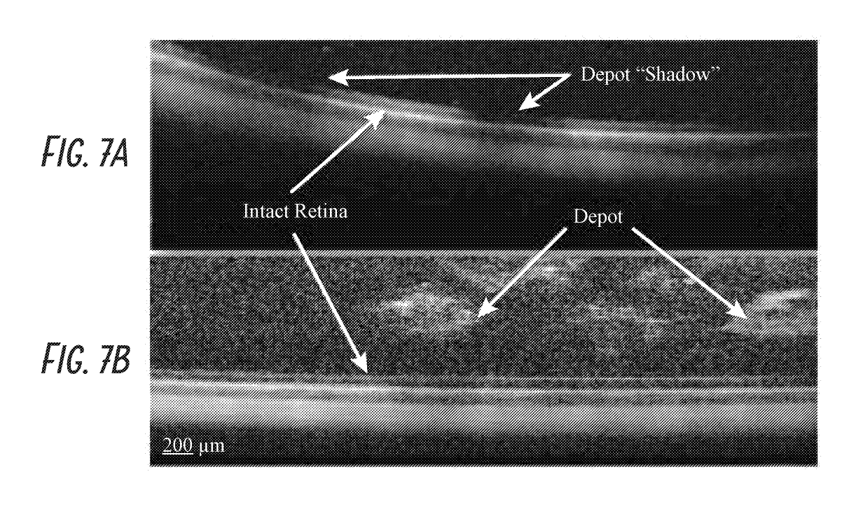

FIG. 7 shows OCT images for placebo and bevacizumab loaded depots that indicate good biocompatibility with the vitreous and rational tissues. No retinal abnormalities or detachments were noted for the duration of the testing. Also, clinical ocular observations using the McDonald-Shadduck scoring system were normal.

Accordingly, the incorporation of a compound, such as the disclosed urea compounds, can have a sustained release of 4-6 months, or even longer, from hydrogel depots. This can lead to improved patient outcomes by lessening the need for frequent injections.

### Example XXVII

### A Non-GLP Blood Sampling Study of C¹⁴ Urea Following Intravitreal Dose Administration in Female Dutch Belted Rabbits

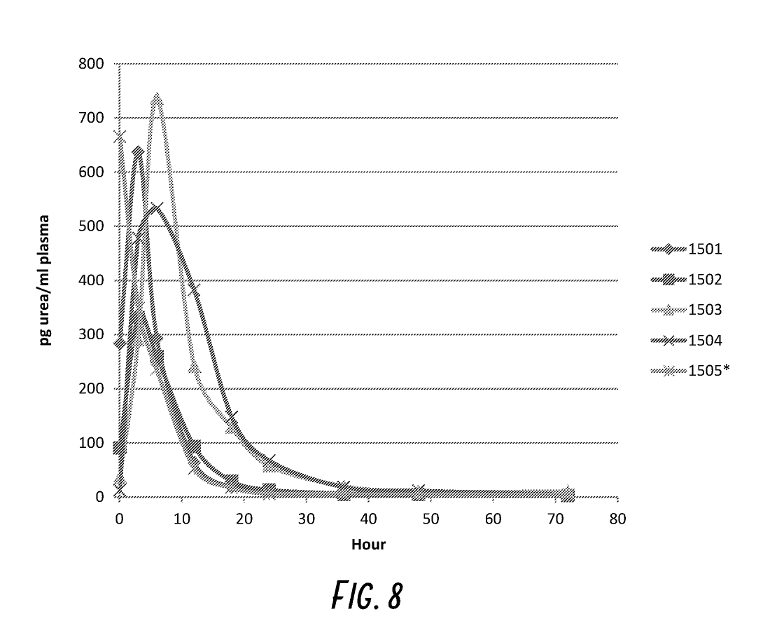

A study was conducted to determine the time course of ¹⁴C Urea in blood following a single intravitreal administration to five (5) female Dutch Belted rabbits. The content of the study is attached hereto as Appendix C, and incorporated herein in its entirety. The PK curve of ¹⁴C-urea in vehicle was determined in five Dutch Belted rabbits (FIG. 8). In the study, ¹⁴C Urea was administered into the left eye of each rabbit as a single intravitreal injection (50 ul dose volume) in 2 mg urea and vehicle (Resolvine). Post injection, blood samples (approximately 2.0 mL of whole blood) were taken from each rabbit at 3 minutes, 3 hours, 6 hours, 12 hours, 18 hours, 24 hours, 36 hours, 48 hours and 72 hours. Plasma samples from the rabbit blood samples were then prepared to determine ¹⁴C Urea levels per ml of plasma. The plasma samples were analyzed using liquid scintillation counting by adding Ecolume to each sample and quantifying beta radiation. The PK curve of ¹⁴C-urea in vehicle for the five (5) Dutch Belted rabbits (rabbits 1501, 1502, 1503, 1504 and 1505) is included as FIG. 8. This study concluded that the Biogel increased the dwell time in the vitreous and retina by approximately 50%.

### Example XXVIII

### Induction of Posterior Vitreous Detachment (PVD) by Multiple Intravitreal Injections of Small Molecule Formulation of Urea

A study was conducted to examine effect of multiple intravitreal injections of small molecule formulation of urea on induction of posterior vitreous detachment (PVD).

Formulation: Sterile lyophilized Urea, USP-NF in buffered isotonic saline for intravitreal injections. Injectable: Reconstituted with 300 μL sterile water for injection. Dosage form: 100 μL dose contains 18 mg Urea, USP-NF (18% w/v) in glass vial. Storage: room temperature. Stability: stable >24 mo for lyophilized form. Safety: up to 4 injections no drug related adverse effects. % resolution of VMA: 44% (non-ERM). PVD rate: 25% targeted efficacy.

U.S. study design: 2:1 randomized, Phase Ib, dose ranging & safety study. Patient population: 30 non-diabetic patients with symptomatic VMA. Treatment: high dose arm (Day 0: 100 μL 18% w/v injection; Day 30: 100 μL 18% w/v injection for all unresolved patients); low dose arm (Day 0: 100 μL 9% w/v injection; Day 30: 100 μL 9% w/v injection for all unresolved patients). Follow-up: days 0, 2, 8, 15, 30, 60, 90 and 180.

Ex-U.S. study design: 3:1 randomized, controlled, Phase Ib, POC & safety study. Patient population: non-diabetic patients with symptomatic VMA. Treatment arm: 20 patients. Control arm: 5 patients. Treatment: high dose arm (Day 0: 100 μL 36% w/v injection; Day 30: 100 μL 36% w/v injection for all unresolved patients); low dose arm (Day 0: 100 μL 18% w/v injection; Day 30: 100 μL 18% w/v injection for all unresolved patients). Follow-up: days 0, 2, 8, 15, 30, 45, 60 and 90.

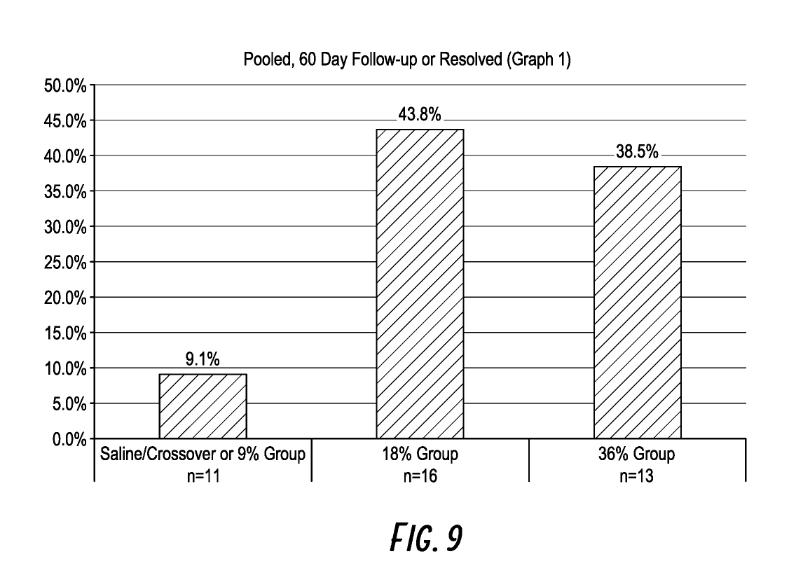

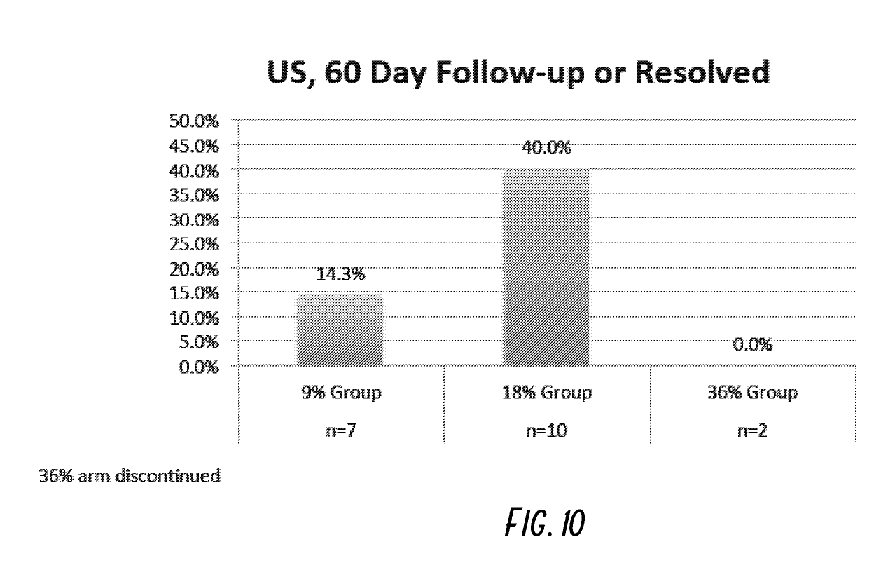

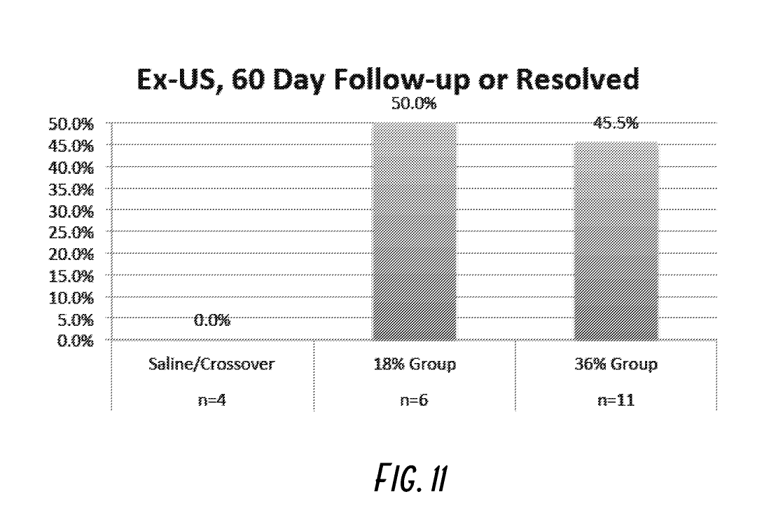

Our results show 30% efficacy with one injection and that increases to 40% after a second injection. FIG. 9 shows the preliminary pooled study results. FIGS. 10 and 11 show the preliminary US and ex-US results, respectively. For the US study, 4 of 10 patients in high-dose group met primary endpoint, 1 of 7 patients in low-dose group met primary endpoint. For the ex-US study, 5 of 11 patients in high-dose group met primary endpoint, 3 of 6 patients in low-dose group met primary endpoint.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

1-29. (canceled)

30. A sustained-release drug delivery system comprising 1.0-99.0% urea and/or urea derivative by weight encapsulated within a biomaterial.

31. The sustained-release drug delivery system of claim 30, comprising an implant.

32. The sustained-release drug delivery system of claim 31, wherein the implant comprises one or more compartments.

33. The sustained-release drug delivery system of claim 32, wherein the urea and/or urea derivative and the biomaterial are contained in the one or more compartments.

34. The sustained-release drug delivery system of claim 30, wherein the biomaterial comprises a polymer.

35. The sustained-release drug delivery system of claim 34, wherein the urea and the polymer form a composite material.

36. The sustained-release drug delivery system of claim 35, wherein the composite material comprises a microparticle or nanoparticle.

37. The sustained-release drug delivery system of claim 36, wherein the microparticle or nanoparticle forms multiple layers.

38. The sustained-release drug delivery system of claim 37, wherein the layers are separated by urea solution and/or crystalline.

39. The sustained-release drug delivery system of claim 36, wherein the microparticle or nanoparticle comprises a hydrogel.

40. The sustained-release drug delivery system of claim 39, wherein the hydrogel is a PEG-chitosan hydrogel.

41. The sustained-release drug delivery system of claim 30, comprising 10-90% urea and/or urea derivative by weight.

42. The sustained-release drug delivery system of claim 30, comprising 10-60% urea and/or urea derivative by weight.

43. The sustained-release drug delivery system of claim 30, wherein the sustained-release drug delivery system is used to treat an eye disorder selected from the group consisting of VMA, PVD, diabetic retinopathy, diabetic macular edema, and wet macular degeneration.

44. A method of treating an eye disorder, comprising administering the sustained-release drug delivery system of claim 30 to a subject.

45. The method of claim 44, wherein the sustained-release drug delivery system releases the urea and/or urea derivative for at least 6 months for a single administration.

46. A pharmaceutical composition for injection into a vitreous body of an eye, comprising a sterile aqueous solution, comprising:
a) 1.0-99.0% urea by weight;
b) 0.00001-1.0% citric acid by weight;
c) 0.05-3.6% sodium chloride by weight; and
d) sterile water for injection, Q.S. 100% by weight;
wherein said pharmaceutical composition is stable and non-toxic when injected into the eye; and
wherein said pharmaceutical composition is retained within a hydrogel thereby providing extended release.

47. A method of treating an eye disorder, comprising:
injecting into the vitreous body a composition comprising an extended release hydrogel, comprising PEG and 1.0-99.0% urea by weight.

48. The method of claim 47, wherein the eye disorder is selected from VMA, PVD, diabetic retinopathy, diabetic macular edema, and wet macular degeneration.

49. The method of claim 45, wherein the composition forms a microencapsulation.
